(19) United States
(12) Patent Application Publication
(10) Pub. No.: US 2020/0216918 A1
(43) Pub. Date: Jul. 9, 2020

(54) FLUORESCENT ENVELOPED VIRAL PARTICLES AS STANDARDS FOR NANOSALE FLOW CYTOMETRY

(71) Applicant: University of Ottawa, Ottawa (CA)

(72) Inventors: Marc-Andre LANGLOIS, Ottawa (CA); Vera TANG, Ottawa (CA); Tyler Milston RENNER, Manotick (CA)

(21) Appl. No.: 16/650,895

(22) PCT Filed: Apr. 27, 2018
(86) PCT No.: PCT/CA2018/000080

§ 371 Date: Mar. 26, 2020

(51) Int. Cl. C12Q 1/6897; G01N 15/10; G01N 15/14; C12Q 1/70; C12N 15/86

(52) U.S. Cl. CPC C12Q 1/6897; G01N 15/1012; G01N 15/14; G01N 2015/0038; C12N 15/86; C07K 2319/60; C12Q 1/702

(57) ABSTRACT

Described herein are uses of fluorescent enveloped virus particles as standards in nanoscale flow cytometry applications. The virus particles comprise a fluorescent dye or a fluorescent protein. A standard ladder comprising a plurality of fluorescent enveloped virus particles of different sizes is also provided. The standards may also comprise marker(s) of interest, and may be used as controls for detection of other viruses or extracellular vesicle, e.g. in diagnostic applications. Methods of producing controls for such applications are provided, including those having desired profiles. The controls may be used for enumeration of markers on microparticles (e.g. extracellular vesicles or viruses). Also described is a modified gammaretrovirus comprising a mutation that abolishes expression of the viral glyco-Gag protein, and having a fluorescent protein inserted in-frame into the proline-rich region (PRR) of the viral env protein. The gammaretrovirus may be M-MLV bearing a mutation that abolishes expression of the glyco-Gal protein, gPr80.

### CROSS REFERENCE TO RELATED APPLICATIONS

This application claims the benefit of priority of U.S. Provisional Patent Application No. 62/563,957 entitled FLUORESCENT ENVELOPED VIRAL PARTICLES AS STANDARDS FOR NANOSALE FLOW CYTOMETRY and filed on Sep. 29, 2017, which is hereby incorporated by reference.

### BACKGROUND

Nanoscale flow cytometry (NFC), also called flow virometry, is an adaptation of flow cytometry technology for the analysis of individual submicron-sized particles.

Size and fluorescence standards are essential for the set-up, optimization, and quality control of flow cytometers. These calibration standards are critical for standardized data acquisition between laboratories, instruments, and technological platforms.

There are currently no size standards that accurately portray the refractive indices of extracellular vesicles (EVs) and viruses, or the level of fluorescence that is achievable on biological particles. As shown in FIG. 1, the apparent size distribution of commercial fluorescent beads displayed as fluorescence intensity as a function of Forward Scatter (FSC) is not uniform for a given size. Current size standards are made of either silica or polystyrene. These materials have a higher refractive index than cellular components, and thus translates into higher scatter values in comparison to biological particles of the same size (FIG. 1, Panel (B)).

Using synthetic bead standards as a tool to measure relative particle size by side scattered light (SSC) or by forward scattered light (FSC) results in a significant underestimation of the actual size of the biological particles when analyzed by flow cytometry.

The analysis of particles in the nanometer size range (e.g., 90-200 nm) is challenging because the size of the particles is at the limit of detection of current flow cytometers. In fact, published specifications for cytometers indicate that 200 nm is the limit of detection. In practice, however, this detection limit is lower, but extensive optimization of assays, and careful set-up and calibration of the cytometers are required to achieve this range.

It would therefore be desirable to develop standards for NFC.

### SUMMARY

It is an object of the present disclosure to obviate or mitigate at least one disadvantage of previous approaches.

In a first aspect, the present disclosure provides a recombinant nucleic acid encoding a modified gammaretrovirus the recombinant nucleic acid comprising: a mutation that reduces or abolishes expression of the viral glyco-Gag protein, and a nucleic acid encoding a fluorescent protein inserted in-frame into the proline-rich region (PRR) of the viral env protein.

In a second aspect, there is provided a use of enveloped virus particles, wherein the virus particles are fluorescent, as a size or calibration standard in nanoscale flow cytometry.

In a third aspect, there is provide a method of calibrating a flow cytometer comprising: measuring a calibration standard comprising enveloped virus particles, wherein the virus particles are fluorescent, in nanoscale flow cytometry.

In a fourth aspect, there is provided a flow cytometry method comprising: measuring a size standard comprising enveloped virus particles, wherein the virus particles are fluorescent, in nanoscale flow cytometry.

In another aspect, there is provided a method of detecting particles by nanoscale flow cytometry, the method comprising calibrating a flow cytometer with a control sample comprising enveloped virus particles as described herein, and, following calibration, detecting particles in a sample by nanoscale flow cytometry.

In another aspect, there is provided a method of detecting viral particles or extracellular vesicles comprising at least one marker, the method comprising calibrating a flow cytometer with a control sample comprising enveloped virus particles as described herein, and detecting viral particles or extracellular vesicles comprising the at least one marker in a sample by nanoscale flow cytometry.

In another aspect, there is provided a method of enumerating markers on microparticles by nanoscale flow cytometry, the method comprising calibrating a flow cytometer with a control sample comprising enveloped virus particles as described herein, and, following calibration, enumerating markers on microparticles of a sample by nanoscale flow cytometry.

In another aspect, there if provided a method of enumerating markers on viral particles or extracellular vesicles, the method comprising calibrating a flow cytometer with a control sample comprising enveloped virus particles as described herein, and enumerating markers on viral particles or extracellular vesicles in a sample by nanoscale flow cytometry.

In another aspect, there is provided a size standard or calibration ladder for nanoscale flow cytometry, comprising a plurality of types of enveloped virus particles, each of the enveloped particles being fluorescent, wherein each of the types of virus particles is of a different size.

In another aspect, there is provided a method of producing fluorescent enveloped virus particles comprising at least one selected marker, the method comprising: infecting a host cell expressing the at least one selected marker with enveloped virus particles, and recovering enveloped virus particles produced by the infected host cell, wherein the recovered enveloped virus particles comprise the at least one selected marker, and wherein the recovered enveloped virus particles are fluorescent.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 (Prior Art) depicts: (A) A scatterplot showing the apparent size distribution of commercial fluorescent beads displayed as fluorescence intensity as a function of Forward Scatter (FSC). (B) Relative FSC mean fluorescence intensity (MFI) in respect to bead manufacturer, reported size, and composition. (C) Refractive indices of VV, whole cells, cellular components, polystyrene and silica beads. (D) Size estimation of VV using polystyrene and silica beads. Linear regression was used to calculate VV size values for both types of beads. (Figure adapted from Tang, VA et al Vaccine. 2016 Sep. 30; 34(42):5082-9.)

FIG. 2 depicts results of analysis of MLVeGFP virions by NFC cytometry. MLVeGFP from infected cell supernatants were filtered through 0.45 μm, 0.2 μm and 0.1 μm pore-sized PES cartridge filters. eGFP+ particles are depicted in green and background noise and/or non-fluorescent particles in black. The number of eGFP+ events in the top right quadrant gate are indicated in green.

FIG. 3 depicts eGFP+ particle count post filtration as a percentage of the total eGFP+ particles in the unfiltered virus preparation.

FIG. 4 shows 293T cells were transfected with expression plasmids for eGFP or Env-eGFP, or with the MLVeGFP expression plasmid. eGFP+ particles were analyzed by NFC as above. Transfection efficiency was approximately 75% in all conditions.

FIG. 5 shows tabulation of average particle counts with S.D for eGFP+ particles from the three independent transfections experiments from FIG. 4.

FIG. 6 shows eGFP expression in EVs (eGFP and Env-eGFP) and MLVeGFP virus was abrogated by treatment with 0.05% Triton.

FIG. 7 shows SSC intensity comparison of eGFP+ EVs and virus shows that MLVeGFP displays a more homogenous particle population that scatters light more intensely.

FIG. 8 shows quantification of SSC intensities (MFI) from three independent experiments with S.D., as described in F. P values were calculated by Student t test.

FIG. 9 shows representative size profiles of particles released from transfected 293T cells expressing eGFP, Env-eGFP, and MLV-eGFP. Samples were analyzed by nanoparticle tracking analysis (NTA). Results are displayed as the percentage of particles within 25 nm segments.

FIG. 10 depicts the effects of fluorescence and SSC thresholding on particle counts and electronic aborts. Effects of sample dilutions and flow rates on data acquisition. MLVeGFP was analysed with the event threshold set at a fluorescence intensity of 500 on green fluorescence channel (488-530/30) (FL-Threshold).

FIG. 11 also depicts effects of fluorescence and SSC thresholding on particle counts and electronic aborts, showing SSC from the 488 nm laser at a fluorescence intensity of 200 (SSC-Threshold). Serial dilutions were performed on 0.45 μm-filtered cell supernatants: undiluted: 1; ½: 0.5; ⅕: 0.2; 1/10: 0.1; 1/20: 0.05; 1/50: 0.02; 1/100: 0.01. All diluted samples were analyzed with the sample flow rate set to low. Undiluted samples were analyzed with flow rates set to low, medium (med) and high.

FIG. 12 (C) Electronic aborts, Total eGFP events and median fluorescence intensity (MFI) of eGFP+ particles were plotted as function of the dilution factor and flow rate. SSC-threshold (dotted line) and FL-threshold (solid line).

FIG. 13 depicts total counts of eGFP+particles plotted for SSC-thresholding against a linear regression plot of predicted events (grey area) based on counts measured for the 1/100 dilution for each thresholding condition. FL-thresholding.

FIG. 14 depicts total counts of eGFP+particles plotted for SSC-thresholding against a linear regression plot of predicted events (grey area) based on counts measured for the 1/100 dilution for each thresholding condition.

FIG. 15 depicts the effects of laser power and PMT voltages on fluorescence and SSC. MLVeGFP was analysed with systematic adjustments of voltage for detection of green fluorescence and SSC off the 488 nm laser. Increments of 50V were made on SSC off the 488 nm laser from 225V to 425V while maintaining a consistent MFI for the eGFP+ population on the green fluorescence channel (488nm-530/30), while simultaneously increasing laser power from 50 to 300 mW. Threshold was set at SSC fluorescence intensity 200.

FIG. 16 depicts results for the same voltage and laser power adjustments made on the green fluorescence channel while maintaining constant the MFI in SSC of the eGFP+ population.

FIG. 17 shows tabulation of the number of eGFP+ events in gates set in FIG. 15.

FIG. 18 shows tabulation of the number of electronic aborts generated during the acquisition of each condition described in FIG. 15.

FIG. 19 Fluorescence Index calculated for each condition portrayed in B. FL-index=(MFI_{eGF+}-MFI_{eGFP-})/SD_{eGFP-}.

FIG. 20 depicts direct staining of MLVeGFP viruses from infected cell supernatants labeled with (A) DiD.

FIG. 21 depicts direct staining of MLVeGFP viruses from infected cell supernatants labeled with Dil.

FIG. 22 depicts direct staining of MLVeGFP viruses from infected cell supernatants labeled with FM4-64.

FIG. 23 depicts direct staining of MLVeGFP viruses from infected cell supernatants labeled with Rhodamine-1,2-DihexadecanoylPhosphatidylethanolamine (DHPE-Rhodamine).

FIG. 24 depicts direct staining of MLVeGFP viruses from infected cell supernatants labeled with Ceramide-BODIPY TR.

FIG. 25 depicts indirect staining of virus. Supernatants of infected and uninfected cells were labeled with DiD and analyzed by NFC.

FIG. 26 depicts indirect staining of virus. Supernatants of infected and uninfected cells labeled with Dil and analyzed by NFC.

FIG. 27 depicts indirect staining of virus. Supernatants of infected and uninfected cells labeled with FM4-64 and analyzed by NFC.

FIG. 28 depicts indirect staining of virus. Supernatants of infected and uninfected cells labeled with DHPE-Rhodamine and analyzed by NFC.

FIG. 29 depicts indirect staining of virus. Supernatants of infected and uninfected cells labeled with Ceramide-BODIPY TR.

FIG. 30 depicts relative virus infectivity (transducing units (TU) per mL) of particles released from the supernatants of infected cells labeled with the different dyes. Infectivity is presented as the percentage of total number of eGFP+ particles released from unstained virus-infected cells.

FIG. 31 depicts counts of eGFP+ particles released from the supernatants of infected cells labeled with the different dyes. Counts are presented as the percentage of total number of eGFP+ particles released from unstained virus-infected cells.

FIG. 32 depicts NFC results for supernatants from DiD-stained infected and uninfected cells treated with 0.05% Triton to demonstrate the lysis of EVs and the removal of eGFP-expressing envelope on the MLVs.

FIG. 33 depicts discrimination of enveloped viruses from EVs by a combination of membrane and nucleic dyes. Panel (A) show that MLVeGFP was treated with 2% PFA at 80° C. to fix the virus and permeabilise the envelope and capsid, rendering the content of the particles amenable to staining by nucleic acid dyes. MLVeGFP infected cells were stained with DiD. Panel (B) shows the results of direct analysis. Panel (F) shows results of staining with SYBR II following fixation and heat treatment. Panel (D) show results for supernatants from unstained infected cells, which were also treated with SYBR II and analysed. Panel (C) shows a comparison of fluorescent staining intensities between supernatants from infected and uninfected cells for DiD stained cells. Panel (E) shows a comparison of fluorescent staining intensities between supernatants from infected and uninfected cells for SYBR II stained supernatants. Panel (G) shows a histogram and dotplot overlay of dual stained DiD/SYBR II nanoparticles from infected and uninfected cells. Nanoparticles from uninfected cells is depicted in black, and from infected cells in blue.

FIG. 34 shows the results of direct labeling of VV with Dil, followed by SYBR I staining; shown in the second and third panel from the left as single stains, followed by double staining in the fourth (right-most) panel.

FIG. 35 shows that four distinct populations are defined by dual labeling using Dil and SYBR I: 1) SYBR+ Dil−, 2) SYBR+Dil+, 3) SYBR−Dil−, 4) SYBR−Dil+. Relative sizes of nanoparticle populations 1 to 4, as displayed as the MFI of SYBR I in relation to SSC.

FIG. 36 shows scatter and fluorescence intensity of currently available bead standards in comparison to fluorescently labeled enveloped viruses. Enveloped viruses ranging in size from MLVeGFP at 120 nm to Vaccinia at ˜300 nm all expressing green fluorescence in comparison to green fluorescent polystyrene beads ranging in size from 160 nm to 240 nm.

FIG. 37 shows fluorescence intensity of MLVeGFP compared with rainbow calibration particles (8 peak beads). These beads contain populations with 8 different fluorescence intensities. The fluorescence of MLVeGFP is only slightly higher than the dimmest (unlabeled) population in the calibration beads.

FIG. 38 shows that MLVeGFP(CTG) expresses higher green fluorescence than wild-type virus. Panel (A) depicts Wild type Moloney MLV, Panel (B) depicts CTG mutant, and Panel (C) depicts comparison of green fluorescence intensities of the two viruses (right-most peak=mutant).

FIG. 39 shows fluorescence stability post storage of MLVeGFP fixed in PFA at 4° C. Panel (A) depicts MLVeGFP freshly produced, and Panel (B) depicts MLVeGFP after 7 weeks of storage.

FIG. 40 shows the results of Nano Tracking Analysis (NTA), revealing no significant size differences between microparticle populations regardless of virus. The vast majority of particles released from these cells overlap with the size of the viruses analyzed.

FIG. 41 depicts results of experiments involving sample fixation and dialysis.

FIG. 42 depicts result of experiments involving use of stabilizers to maintain particle count and eGFP fluorescence of the virus

FIG. 43 depicts enumeration of antigen expression on the virus surface by two methods.

FIG. 44 depicts results of experiments on use of virus particles to test instrument fluorescence sensitivity on various flow cytometry platforms.

FIG. 45 shows comparison of instrument settings determined using beads (ApogeeMix) with settings optimized with MLVeGFP and ALVsfGFP for analysis of extracellular vesicles by nanoscale flow cytometry.

FIG. 46 depicts particle counts of DiO-labeled extracellular vesicles analyzed using bead and virus optimized setting.

FIG. 47 depicts results of experiments involving use of virus particles as positive controls for extracellular vesicle labeling and surface antigen profiling.

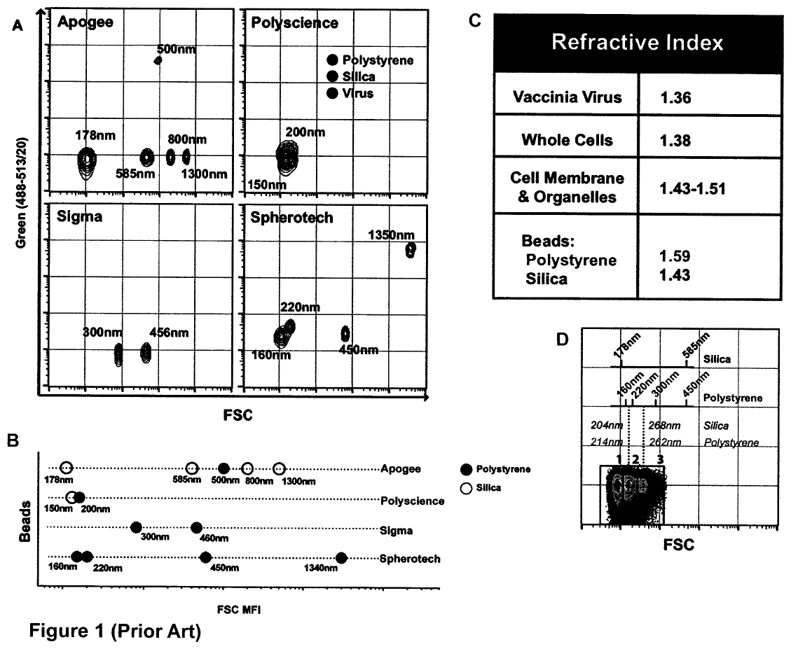

### DETAILED DESCRIPTION

Generally, the present disclosure provides fluorescent enveloped virus particles for use as a size or calibration standard in nanoscale flow cytometry. The standards may be used as controls in some applications.

Also described is a recombinant nucleic acid encoding a modified gammaretrovirus, the recombinant nucleic acid comprising a mutation that abolishes expression of the viral glyco-Gag protein, and a nucleic acid encoding a fluorescent protein inserted in-frame into the proline-rich region (PRR) of the viral env protein.

### Modified M-MLV Encoding Nucleic Acids

In one aspect, there is provided a recombinant nucleic acid encoding a modified gammaretrovirus the recombinant nucleic acid comprising: a mutation that reduces or abolishes expression of the viral glyco-Gag protein, and a nucleic acid encoding a fluorescent protein inserted in-frame into the proline-rich region (PRR) of the viral env protein.

By “gammaretrovirus” will be understood as members of the eponymous genus of the retroviridae family. Examples includes those viruses known as CAS-BR-E, MLV 1313 (Amphotropic MLV), Pmv11 (Polytropic MLV), Xmx15 (Xenotropic MLV), FrMLV (Friend MLV), M-MLV (Moloney MLV), DG-75, AKV MLV (AKV MLV), SL3-3 MLV, E-MLV (Ecotropic MLV), Rauscher MLV, Mus Dunni endogenous virus, Abelson MLV, or XMRV. The term will be understood to encompass relative viruses having, e.g. 80%, 90%, 95%, 98%, or 99% sequence identify to any member of the gammaretrovirus family.

By “recombinant” is meant a nucleic acid that has been genetically altered, e.g., by the addition or insertion of a heterologous nucleic acid that is not present in the natural sequence.

By “mutation that reduces abolishes expression of the viral glyco-Gag protein” is meant any suitable sequence change that results in a relative reduction or reduction to undetectable levels (respectively), of the extended form of Gag, termed glyco-Gal (gPr80), that is characteristic of gammaretroviruses. The viral glyco-gag can be readily identified, e.g. by sequence conversation (e.g., homology) with glyco-Gal (gPr80) of M-MLV.

By “fluorescent protein” is meant a protein that absorbs light of a specific wavelength (e.g., absorption wavelength) and emits light with a longer wavelength (e.g., emission wavelength). The term fluorescent protein encompasses natural fluorescent proteins (i.e., the natural form of the fluorescent protein without any genetic manipulations) and genetically mutated fluorescent proteins (e.g., fluorescent proteins engineered to change the identity of one or more amino acid residues). Fluorescent proteins include, but are not limited to, green fluorescent proteins (e.g., GFP, enhanced GFP (eGFP), Emerald, Superfolder GFP, Azami Green, mWasabi, TagGFP, TurboGFP, AcGFP, ZsGreen, T-Sapphire, and T-Sapphire), blue fluorescent proteins (e.g., EBFP, EBFP2, Azurite, mTagBFP), cyan fluorescent proteins (e.g., ECFP, mECFP, Cerulean, CyPet, AmCyan1, Midori-Ishi Cyan, TagCFP, mTFP1 (Teal)), yellow fluorescent proteins (e.g., EYFP, Topaz, Venus, mCitrine, YPet, TanYFP, PhiYFP, ZsYellow1, and mBanana), orange fluorescent proteins (e.g., Kurabira Orange, Kurabira Orange2, mOrange, mOrange2, dTomato, dTomato-Tandem, TagRFP, TagRFP-T, DsRed, DsRed2, DsRed-Express (T1), DsRed-Monomer, and mTangerine), and red fluorescent proteins (e.g., mRuby, mApple, mStrawberry, AsRed2, mRFP1, JRed, mCherry, HcRed1, mRaspberry, dKeima-Tandem, HcRed-Tandem, mPlum, and AQ143).

In one embodiment, the fluorescent protein is green fluorescent protein (GFP). “GFP” will be understood to refer to the green fluorescent protein from the jellyfish, Aequorea victoria. The fluorescent protein may also be a GFP derivative, for example, comprising one or more mutation that enhances fluorescent relative to the parent GFP. In one embodiment, the fluorescent protein may be enhanced green fluorescent protein (eGFP). “eGFP” will be understood to comprise an F64L mutation, yielding improved characteristics, such as extinction coefficient and quantum yield.

A skilled person would readily appreciate that a nucleic acid encoding a fluorescent protein could comprise sequence changes that do not substantially reduce or abrogate fluorescence. This could be readily tested.

By “inserted” is meant cloned into the proline-rich region in an in-frame manner.

By “in frame” is meant that the coding sequence for the fluorescent protein will be cloned in the same reading frame as the env protein into which it is inserted. The manner of cloning will be understood to permit the fluorescent protein to be expressed and to properly fold and achieve its fluorescent character. The fluorescent protein may be linked by an appropriate spacer.

In one embodiment, the modified gammaretrovirus is CAS-BR-E, MLV 1313 (Amphotropic MLV), Pmv11 (Polytropic MLV), Xmx15 (Xenotropic MLV), FrMLV (Friend MLV), M-MLV (Moloney MLV), DG-75, AKV MLV (AKV MLV), SL3-3 MLV, E-MLV (Ecotropic MLV), Rauscher MLV, Mus Dunni endogenous virus, Abelson MLV, XMRV, Porcine endogenous type C, Gibbon leukemia virus, Baboon endogenous virus strain M7, Feline leukemia virus, Koala retrovirus, or Wooly monkey virus. The recombinant nucleic acid may comprise a nucleic acid encoding one of these viruses, for example, as listed in Table 1 by GenBank Accession Number.

TABLE 1

| | Gammaretrovirus | | GenBank |
|---|---|---|---|
| | Name | Common Name | Accession No. |
| Murine | CAS-BR-E | | M14702.1 |
| | MLV1313 | Amphotropic MLV | AF411814.1 |
| | Pmv11 | Polytropic MLV | KJ668271.1 |
| | Xmx15 | Xenotropic MLV | HQ154630.1 |
| | FrMLV | Friend MLV | U13766.1 |
| | MoMLV | Moloney MLV | NC_001501.1 |
| | DG-75 | | AF221065.1 |
| | AKV MLV | AKV MLV | J01998.1 |
| | SL3-3 MLV | | AF169256.1 |
| | E-MLV | Ecotropic MLV | KJ668270.1 |
| | Rauscher MLV | Rauscher MLV | U94692.1 |
| | | Mus Dunni endogenous | AF053745.1 |
| | | virus | |
| | Abelson MLV | Abelson MLV | AF033812.1 |
| | XMRV | | FR872816.1 |
| Non- | | Procine endogenous type C | EF133960.1 |
| Murine | | Gibbon leukemia virus | U60065.1 |
| | | Baboon endogenous virus | AB979448.1 |
| | | strain M7 | |
| | | Feline leukemia virus | AB672612.1 |
| | | Koala retrovirus | AB721500.1 |
| | | Wooly monkey virus | KT724051.1 |

In one embodiment, the recombinant nucleic acid may comprise a nucleic acid having a sequence that is at least 80% identical to one of the references sequences in Table 1. In one embodiment, the recombinant nucleic acid may comprise a nucleic acid having a sequence that is at least 85% identical to one of the references sequences in Table 1.In one embodiment, the recombinant nucleic acid may comprise a nucleic acid having a sequence that is at least 90% identical to one of the references sequences in Table 1. In one embodiment, the recombinant nucleic acid may comprise a nucleic acid having a sequence that is at least 95% identical to one of the references sequences in Table 1. In one embodiment, the recombinant nucleic acid may comprise a nucleic acid having a sequence that is at least 98% identical to one of the references sequences in Table 1. In one embodiment, the recombinant nucleic acid may comprise a nucleic acid having a sequence that is at least 99% identical to one of the references sequences in Table 1.

In one embodiment, the modified gammaretrovirus is a modified Moloney murine leukemia virus (M-MLV), wherein the mutation reduces or abolishes expression of gPr80 protein.

In one embodiment, the recombinant nucleic acid comprises a nucleic acid sequence derived from GenBank Accession NCBI: NC_001501, modified with said mutation and said nucleic acid encoding fluorescent protein. It will be appreciated that this GenBank entry is annotated, permitting its features to be readily identified. For example, where viral proteins are named with respect to M-MLV, a skilled person would appreciate that these proteins correspond to those annotated in NC_001501.

By “derived from”, a skilled person would appreciate that the recombinant nucleic acid could comprise sequence changes relative to this reference sequence to the extent that they do not negatively impact the assembly the viral particles for the intended application. An example would be silent mutations in coding regions that do not impact coding, transcription, or translation. Conservative amino acid changes (e.g. resulting in amino acids with similar side chain chemistry) could also be encompassed. The recombinant nucleic acid so derived could, for example comprise a sequence that has 80%, 85%, 90%, 91%, 92%, 93%, 94%, 95%, 96%, 97%, 98%, 99% sequence identity to NC_001501 across the full length thereof. The recombinant nucleic acid so derived could encode the proteins encoded by NC_001501, with or without amino acid sequence differences.

In one embodiment, the recombinant nucleic acid comprises the nucleic acid sequence of GenBank Accession NCBI: NC_001501, modified with said mutation and said nucleic acid encoding fluorescent protein.

In one embodiment, the mutation that reduces or abolishes expression of gPr80 is in the CTG codon at positions 93-95 of NC_001501. In one embodiment, the mutation that reduces abolishes expression of gPr80 is a CTG→CTA mutation. It has surprisingly been found that such a mutation increases the fluorescence of M-MLV in which the env protein is labelled with eGFP. Without being bound by theory, it is believed that reduction abrogation of gPr80 expression from the CTG alternate start codon leads to more expression of env-eGFP, and/or greater incorporation of the env-eGFP into the viral particles. It is expected that other sequences change to reduce or abrogate translation from the CTG at positions 93-95 (or otherwise reduce expression of gPr80) would produce similar effects. Likewise, it is expected that mutations in other gammaretroviruses that reduce or abolish expression of the glyco Gal-pol would have similar effects. These mutation may be in the start codon from which transcription of the glyco Gal-pol is initiated. These mutations may be in the codon corresponding to positions 93-95 of M-MLV, which would be readily identifiable, e.g., by sequence alignment.

By “proline-rich region” will be understood the flexible area of the viral env protein. The proline rich region corresponds to the region encoded by nucleotide positions 6299 to 6435 of NC_001501. For other gammaretroviruses, a corresponding region or position for insertion could be readily identified by sequence alignment with NC_001501.

In one embodiment, the PRR of the viral env protein corresponds to the region encoded by nucleotide positions 6302 to 6433 of GenBank Accession NCBI: NC_001501.

In one embodiment, the fluorescent protein is inserted into the PRR after the serine at position 6400.

In one embodiment, there is provided a vector comprising the above-described nucleic acid.

By “vector” is meant any genetic element, such as a plasmid, phage, transposon, cosmid, chromosome, virus, virion, etc., which is capable of replication when associated with the proper control elements and which can transfer gene sequences into or between cells. Thus, the term includes cloning and expression vehicles, as well as viral vectors.

In one aspect, there is provided a host cell comprising the above-defined nucleic acid or the above-defined vector.

By “host cell” is meant a cell or population of cells into which the recombinant nucleic acid has been introduced. In certain embodiments, a host cell of the present invention is a eukaryotic cell or cell line, for example, a plant, animal, vertebrate, mammalian, rodent, mouse, primate, or human cell or cell line. Any suitable host cells which will support propagation of or expression from a given recombinant nucleic acid is intended. The host cell may be, e.g., transiently or stably transfected, or infected such that it comprises the nucleic acid or vector. The recombinant nucleic acid may be integrated into the genome of the host cell.

In one embodiment, the host cell comprises at least one selected marker. The purpose of having a host cell comprising a marker, in some embodiments, is to facilitate uptake of the at least one selected marker by viruses that egress from the host cell, thus permitting recovery of viral particles comprising the at least one selected marker.

By “marker” will be understood any molecule or protein whose presence can be detected. Such a molecule may be detectable, e.g. by immunological methods (e.g. antibodies) or by staining. The markers may be part of, associated with, or otherwise derived from any membrane of a cell, such as the outer cell membrane or an intracellular membrane.

Examples of the latter include organelle and vesicle membranes, such as endocytic vesicle membranes. Accordingly, a “marker” may be, e.g. an endocytic marker. Example markers include proteins, phospholipids, glycoproteins, or receptors. Markers may be membrane components, membrane-anchored components, membrane-associated components, membrane-spanning components, or membrane derived components.

In one embodiment, the at least one selected marker may be characteristic of a particular cell type or cellular state. In one embodiment, the at least one selected marker may be operationally specific to a particular cell type or cellular state. “Operationally unique” is intended to mean distinguishable in the context of the sample with the detection means employed. In one embodiment, the at least one selected marker may be unique to a particular cell type or cellular state.

In one embodiment, the at least one selected marker comprises a plurality of markers of a selected profile. By “profile” will be understood a particular set of markers.

In one embodiment, the selected profile may be characteristic of a particular cell type or cellular state. In one embodiment, the selected profile may be operationally specific to a particular cell type or cellular state. In one embodiment, the selected profile may be unique to a particular cell type or cellular state.

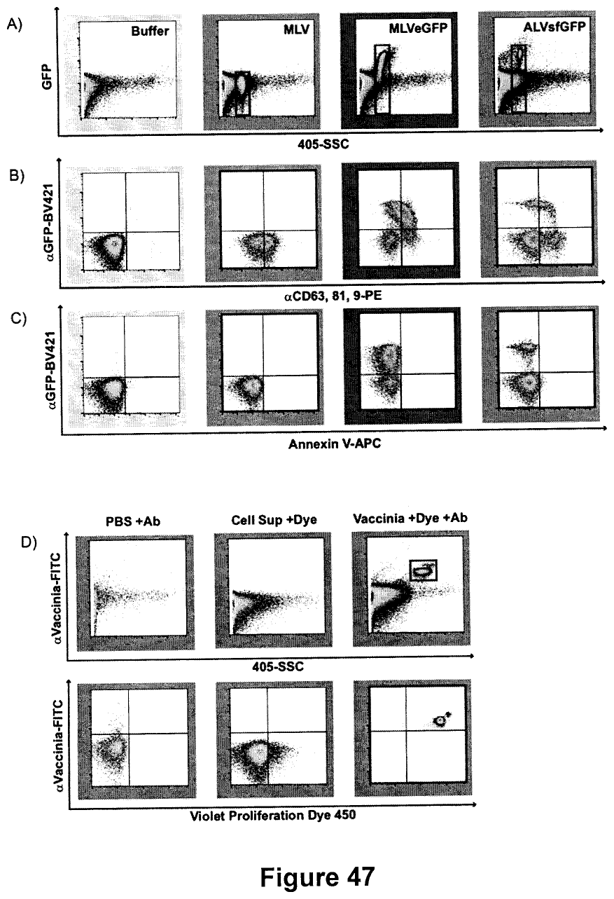

By employing host cells that comprise the at least one marker, viral particles that egress from the host cell may take up the at least one marker. As discussed, the at least one marker need not be limited to markers that are part of or associated with the outer membrane. The cell-derived envelope of enveloped viruses may also, in some instances, comprise, e.g., endocytic vesicle membranes, and thereby may comprise proteins and/or lipids found therein. This is a result of their release through the endocytic secretion pathways shared by exosomes. Accordingly, viruses may display endocytic makers on their surface (e.g., see. tetraspanins CD9, CD63 and CD81—see FIG. 47). Viral particles thus derived may prove useful as controls in the detection of other viral particles or EVs comprising the at least one selected marker. For example, the controls and the viral particles or EVs to be detect may comprise the same at least one marker. The controls and the viral particles or EVs to be detect may comprise the same profile of markers.

The host cell type may be established based on a marker of interest already being expressed by that cell type (some applications may involve pseudotyping a virus in order to permit infection of the desired host cell). That is to say, the marker may be endogenous to the host cell in some embodiments. The marker may be expressed endogenously by the host cell due to a cellular state, such as a disease state.

In other embodiments, the host cell may be modified (e.g. transfected, infected, or otherwise manipulated, e.g. by CRISPR) to express an exogenous marker of interest. The host cell may be recombinant.

In some embodiments, the at least one marker may be modified to increase incorporation into the viral envelope upon egress, in some embodiments. For example, the at least one marker may be a recombinant protein comprising a transmembrane (TM) domain of the native viral envelope glycoprotein. In other embodiments, the marker may be modified with a membrane signal peptide In order to direct the insertion of a protein into a membrane and increase incorporation into viral particles upon egress.

In some embodiments, the at least one selected marker(s) may be characteristic of disease cells. Here, “disease cell” will be understood to indicate a cell in anything other than a healthy state. For example, the host cell may be infected with a pathogen, may be in state of inflammation, may be in an altered metabolic state, may be undergoing apoptosis, may be a pre-cancerous cell, or may be a cancer cell. The marker(s) may indicate the presence, stage, or severity of disease; or may provide information about the affected cell type (e.g., the location of an infection).

Cells of a subject could be analyzed to determine a characteristic marker or profile thereof, in order to select the at least one marker or the profile. Controls could then be generated.

In one aspect, there is provided an enveloped virus particle produced by the above-described cell.

By “enveloped virus”, as used herein, is meant a virus that has an envelope covering its protective protein capsids. Envelopes are typically derived from portions of the host cell membranes (phospholipids and proteins), but comprise some viral glycoproteins.

They may help viruses avoid the host immune system. Glycoproteins on the surface of the envelope serve to identify and bind to receptor sites on the host's membrane. The viral envelope then fuses with the host's membrane, allowing the capsid and viral genome to enter and infect the host. Non-limiting examples of enveloped virus include Herpesviruses, Poxviruses, Hepadnaviruses, Flavivirus, Togavirus, Coronavirus, Hepatitis D, Orthomyxovirus, Paramyxovirus, Rhabdovirus, Bunyavirus, Filovirus, and Retroviruses.

In one embodiment, the enveloped virus comprises the at least one selected marker.

In one embodiment, the enveloped virus comprises the plurality of markers of the selected profile.

In some embodiments, the marker(s) may be recombinant. For example, the marker(s) may be modified to increase incorporation into the viral particles, e.g. as described above.

### Size/Calibration Standards in NFC

In one aspect, there is provided a use of enveloped virus particles, wherein the virus particles are fluorescent, as a size or calibration standard in flow cytometry.

In one aspect, there are provided enveloped virus particles for use as a size or calibration standard in nanoscale flow cytometry, wherein the virus particles are fluorescent.

In one embodiment, the flow cytometry is nanoscale flow cytometry.

By ““nanoscale flow cytometry” is meant flow cytometry as applied to particles of less than 1 μm in size. The technology may be used to analyze biological molecules such as proteins, DNA and lipids inside or on the surface of individual cells. Cell components labeled with fluorescent antibodies or fluorescent reporter proteins, such as eGFP, are excited by a laser to emit light, which is then detected and its intensity measured by the flow cytometer. This provides highly quantitative information on the relative abundance of these molecules in a single cell. With advances in optics, fluidics and laser technologies, the most powerful commercial flow cytometers can now be configured and optimized to analyze microparticles, EVs and viruses in an approach called nanoscale flow cytometry or NanoFlow. This technology can provide a wealth of information on the relative sizes of EVs in a sample, distribution of sub-populations, and identify the specific protein and lipid markers on their surface. Furthermore, cell sorters with upgraded lasers and optics can also sort and isolate microparticles based on size and or fluorescence. However, most commercial flow cytometers do not come pre-configured to analyze microparticles. Hardware modifications, adjustments and optimizations may be required, as well as adaptations to, e.g., sample preparation and staining procedures for the challenges of microparticle analyses.

By “standard” will be understood a sample provided to serve as a reference. The standard may possess a pre-determined property, such as size and/or fluorescence intensity. The standard may be used to calibrate equipment, or may be run with or in parallel to a test sample, e.g., to provide a reference or benchmark. Some standards may comprise a plurality of types of enveloped virus particles, e.g. having different sizes. A standard may be designed or selected based on the nature of the test samples.

By “microparticle” is meant any particle that is less than 1 μm in size. This term can include living organisms such as small bacteria or cells, or particles that are secreted or released at the surface of cells through budding, such as extracellular vesicles.

“Extracellular vesicle” (EV) is a term used to describe small biological structures with a membranous outer layer that are released from the cells of all three domains of life: Archea, Bacteria and Eukaryota. EVs that bud from bacteria are surrounded by the same components that constitute the cell wall, such as peptidoglycans and lipopolysaccharides; whereas EVs released from eukaryotic cells are enveloped by a bilayer phospholipid structure. There are three types of submicron-sized EVs that are known to be released from eukaryotic cells: 1) microvesicles (MVs) (50 nm-1000 nm); 2) exosomes (60 nm-100nm), and 3) enveloped viruses and virus-like particles (60 nm-300 nm).

“Microvesicles” (MVs) are structures released at the plasma membrane that contain elements of the cytosol such as lipids, proteins, mRNAs and micro RNAs. The release of MVs is a natural and continuous process carried out by all types of cells. EVs carry the same surface receptors, markers and antigens as the cell from which they are released. MVs can be taken up by a recipient cell by several ways, including receptor-mediated fusion with the plasma membrane at the surface of a cell, non-specific uptake through phagocytosis or macropinocytosis, and through clathrin-, calveolin-, or lipid raft-mediated endocytosis followed by fusion with the endosomal membrane. Once the cargo of MVs is released, these molecules can alter biological functions in the recipient cell. For example, cytokines can be released that will activate the transcription of certain genes involved in immune defenses against a specific pathogen, proteins can be expressed from cargo mRNA that will induce the cell to proliferate, and miRNAs can downregulate the expression of specific proteins. These subtle modifications to a recipient cell's metabolism constitute a way for cells to communicate information and harmonize responses with cells both near and far.

“Exosomes” are also extracellular vesicles surrounded by a phospholipid bilayer which, from a biochemical standpoint, are undistinguishable from MVs. Although they are on average smaller than MVs, they also contain cell surface receptors and cytosolic proteins, lipids, mRNAs and miRNAs. The defining feature that distinguishes MVs from exosomes is their cellular origin and mode of secretion. While MVs are released by budding at the cell surface, exosomes originate from intraluminal vesicles (ILVs) located inside multivesicular bodies. Multivesicular bodies, or late endosomes, are formed by the internalization of the plasma membrane, and therefore contain cell surface markers and cytosolic components. This is where proteins are sorted before being either permanently disassembled by degradative lysosomes, or trafficked to the cell surface where ILVs are released into the extracellular space as exosomes. There are several mechanisms that have been identified that recruit specific RNAs and proteins into ILVs. This is why exosomes are often enriched in certain types of RNAs.

In one embodiment, the nanoscale flow cytometry is for measurement of particles less than 1 μm, 900 nm, 800 nm, 700 nm, 600 nm, 500 nm, 400 nm, or 300 nm in size. In one embodiment, the nanoscale flow cytometry is for measurement of particles less than 250 nm in size. In one embodiment, the nanoscale flow cytometry is for measurement of particles less than 200 nm in size. In one embodiment, the nanoscale flow cytometry is for measurement of particles less than 120 nm in size. In one embodiment, the nanoscale flow cytometry is for measurement of particles less than 100 nm in size. In one embodiment, the nanoscale flow cytometry is for measurement of particles less than 50 nm in size. In one embodiment, the particles are from 10 nm to 200 nm in size. In one embodiment, the particles are from 25 nm to 200 nm in size. In one embodiment, the particles are from 50 nm to 200 nm in size. In one embodiment, the particles are from 120 nm to 200 nm in size. In one embodiment, the particles are from 90 nm to 200 nm in size. In one embodiment, the particles are from 100 nm to 200 nm in size. In one embodiment, the particles are from 10 nm to 200 nm in size. In one embodiment, the particles are from 25 nm to 250 nm in size. In one embodiment, the particles are from 50 nm to 250 nm in size. In one embodiment, the particles are from 90 nm to 250 nm in size. In one embodiment, the particles are from 100 nm to 250 nm in size. In one embodiment, the particles are from 120 nm to 250 nm in size. In one embodiment, the particles are from 10 nm to 500 nm in size. In one embodiment, the particles are from 25 nm to 500 nm in size. In one embodiment, the particles are from 50 nm to 500 nm in size. In one embodiment, the particles are from 90 nm to 500 nm in size. In one embodiment, the particles are from 100 nm to 500 nm in size. In one embodiment, the particles are from 120 nm to 500 nm in size. In one embodiment, the particles are from 90 nm to 120 nm in size.

In one embodiment, the enveloped virus particles each comprise fluorescent dye. The dye may be a lipid-targeting dye, such as a lipid membrane-targeting dye. Examples include DID, Dil, FM4-64, fluorescent sphingolipid (Ceramide-BODIPY TR), or fluorescent phospholipid (DHPE-Rhodamine). The dye may be a protein-targeting dye. The dye may be a nucleic acid targeting dye. Examples include SYBR II (for RNA) or SYBR I (for DNA). Enveloped virus particles can be either indirectly or directly labeled with a fluorescent dye. In one embodiment, the enveloped virus particles are indirectly labelled with the fluorescent dye. “Indirect labelling” is done by staining a virus-infected cell with a fluorescent dye. Proteins and/or lipids in this cell become stained, and egress virus particles then take-up the dye by acquiring labeled proteins and lipids. In one embodiment, the enveloped virus particles are directly labelled with the fluorescent dye. “Direct labelling” is done by purifying egress virus and adding a dye directly to them. These methods of labeling viruses don't appear to be clearly defined.

In on embodiment, the enveloped virus particles each comprise one or more fluorescent protein. In one embodiment, the fluorescent protein is green fluorescent protein (GFP). In one embodiment, the fluorescent protein is enhanced green fluorescent protein (eGFP). In one embodiment, the enveloped virus particles each comprise viral envelope proteins, each labelled with the fluorescent protein.

By “labelled” it will be understood that the viral envelope protein is somehow attached, coupled, or linked to the fluorescent protein. This could be achieved, e.g., by chemical addition, or by modification of the envelope protein coding nucleic acid sequence so that it is linked to (or contains) the in-frame coding sequence for the fluorescent protein. The fluorescent protein would thus be translated and would fold to recapitulate the fluorescence of the parent protein from which it is determined. The linking could be done via a suitable spacer sequence, as necessary.

In one embodiment, each of the envelope proteins is labelled with one fluorescent protein.

In one embodiment, the viral envelope proteins are M-MLV envelope proteins.

In one embodiment, the enveloped virus particles are pseudotyped with the viral envelope proteins.

By “pseudotyped” will be understood that the enveloped virus is produced such that it incorporates a non-native or modified envelope protein. This method can be used to alter host tropism or to achieve an increased/decreased stability of the virus particles. For example, cell infected with a particular enveloped virus could be further modified to comprise a sequence encoding a pseudotyping construct expressing the non-native or modified envelope protein.

In some embodiments of the present invention, the pseudotyping is accomplished with an M-MLV envelope protein, e.g. linked to eGFP (env-eGFP), as described herein. This has been found to particularly effective for fluorescent labelling of viral particles.

In one embodiment, the viral envelope protein and the fluorescent protein are as encoded by SEQ ID NO: 1. SEQ ID NO: 1 depicts the nucleic acid coding sequence (CDS) according to one embodiment. In one embodiment, the pseudotyping is accomplished with a vector comprising the sequence of SEQ ID NO: 2. SEQ ID NO: 2 depicts the nucleic acid sequence of a vector for pseudotyping, according to one embodiment.

In one embodiment there is provided a use of a nucleic acid comprising SEQ ID NO: 1 or SEQ ID NO: 2 for pseudotyping an enveloped viral particle.

In one embodiment there is provided a method of pseudotyping an enveloped viral particle, comprising introducing, into a cell infected with an enveloped virus, a nucleic acid comprising SEQ ID NO: 1 or SEQ ID NO: 2.

In one embodiment there is provided a method of pseudotyping an enveloped viral particle, comprising introducing, into a cell comprising sequences for producing an enveloped virus, a nucleic acid comprising SEQ ID NO: 1 or SEQ ID NO: 2.

In one embodiment, the enveloped virus particles are pseudotyped to be non-infectious to humans.

In one embodiment, the enveloped virus particles are encoded by a nucleic acid comprising at least one sequence modification that reduces or abrogates expression of the endogenous viral envelope protein. Reduction of expression of endogenous viral envelope protein renders pseudotyping with a non-endogenous viral envelop protein more efficient.

The enveloped virus particles could be of any suitable virus type. In one embodiment, the enveloped virus particles comprise MLV, ALV, HIV, HSV, Influenza, and/or VSV particles. In one embodiment, the virus particles are gammaretrovirus particles. In one embodiment, the gammaretrovirus particles are CAS-BR-E, MLV 1313 (Amphotropic MLV), Pmv11 (Polytropic MLV), Xmx15 (Xenotropic MLV), FrMLV(Friend MLV), M-MLV (Moloney MLV), DG-75, AKV MLV (AKV MLV), SL3-3 MLV, E-MLV (Ecotropic MLV), Rauscher MLV, Mus Dunni endogenous virus, Abelson MLV, XMRV, Porcine endogenous type C, Gibbon leukemia virus, Baboon endogenous virus strain M7, Feline leukemia virus, Koala retrovirus, or Wooly monkey virus. In one embodiment, the enveloped virus particles comprise M-MLV particles.

In one embodiment, the enveloped virus particles are modified M-MLV particles encoded the recombinant nucleic acid as defined above.

In one embodiment, the enveloped virus particles comprise a plurality of enveloped virus types, each of the types being a different size.

By “different size”, in this context, will be understood as distinguishable as different sizes, e.g., as determined by cryo-electron microscopy (cryo-EM). Cryo-EM methods are described, for example, in by Yeager et al. (1998).⁶⁷

In one embodiment, the enveloped virus particles are of a single virus type.

In one embodiment, the enveloped virus comprises at least one selected marker (e.g., as defined above). In one embodiment, the at least one selected marker may be characteristic of a biological parameter. In one embodiment, the at least one selected marker may be characteristic of a particular cell type or cellular state. In one embodiment, the at least one selected marker may be operationally specific to a particular cell type or cellular stateln one embodiment, the at least one selected marker may be unique to a particular cell type or cellular state.

In one embodiment, the enveloped virus comprises a plurality of markers of the selected profile (e.g. as defined above). In one embodiment, the selected profile may be characteristic of a biological parameter. In one embodiment, the selected profile may be characteristic of a particular cell type or cellular state. In one embodiment, the selected profile may be operationally specific to a particular cell type or cellular state. In one embodiment, the selected profile may be unique to a particular cell type or cellular state.

In some embodiments, the at least one marker may be modified to increase incorporation into the viral envelope upon egress, in some embodiments. For example, the at least one marker may be a recombinant protein comprising the transmembrane (TM) domain of the native viral envelope glycoprotein. In other embodiments, the marker may be modified with a membrane signal peptide In order to direct the insertion of a protein into a membrane and increase incorporation into viral particles upon egress.

In some embodiments, the at least one selected marker(s) may be characteristic of disease cells. For example, marker may be indicative of infection with a pathogen, a state of inflammation, an altered metabolic state, apoptosis, pre-cancer, or cancer. The marker(s) may indicate the presence, stage, or severity of disease; or may provide information about the affected cell type (e.g., the location of an infection).

In some embodiments, the size or calibration standard is a control for detection of viral particles or extracellular vesicles. In some embodiments, the control may be a negative control. In some embodiments, the control may be positive control. In some embodiments, the positive control may be for the detection of viral particles or extracellular vesicles comprising the same at least one selected marker. In some embodiments, the size or calibration standard is a positive control for detection of viral particles or extracellular vesicles comprising the same plurality of markers of the selected profile.

Where “same” is referred to it in this context will be appreciated that is to be viewed from the perspective of detection. That is to say, viral particles comprising recombinant marker(s) could be used as controls for the detection of viral particles or extracellular vesicles comprising the “same” markers, even if the detected markers are not themselves recombinant.

In one embodiment, the control is for enumeration of markers on microparticles of interest (e.g. EVs or viruses). By comparing to calibrated international control Molecules of Equivalent Soluble Fluorophores (MESF) bead standard having a defined number of florescent particles, e.g., as established by the National Institute of Standards and Technology (NIST), the number of fluorescent markers on viral particles of the control may be established, e.g. by linear regression. Thereafter, the control may be used as an “MESF surrogate” to enumerate markers on microparticles from a sample. For example, in some applications markers on microparticles and the markers on the MESF surrogate control could be labelled with the same fluorophore, e.g. by binding of a fluorescent antibody.

### Flow Cytometry Methods

In one aspect, there is provided a method of calibrating a flow cytometer comprising: measuring a calibration standard comprising enveloped virus particles, wherein the virus particles are fluorescent, in nanoscale flow cytometry.

In one aspect, there is provided a flow cytometry method comprising: measuring a size standard comprising enveloped virus particles, wherein the virus particles are fluorescent, in nanoscale flow cytometry.

In some embodiment, the enveloped virus particles comprise a fluorescent dye.

In some embodiment, the enveloped virus particles comprise a fluorescent protein.

In some embodiments, the step of measuring comprises measuring enveloped virus particles as described above. In some embodiments, the step of measuring comprises measuring enveloped virus particles comprising particles encoded by the recombinant nucleic acid as described above.

In one aspect, there is provided a method of detecting particles by nanoscale flow cytometry, the method comprising calibrating a flow cytometer with a control sample comprising enveloped virus particles as described herein, and, following calibration, detecting particles in a sample by nanoscale flow cytometry.

In one aspect, there is provided a method of detecting viral particles or extracellular vesicles comprising at least one marker, the method comprising calibrating a flow cytometer with a control sample comprising enveloped virus particles as described herein, and detecting viral particles or extracellular vesicles comprising the at least one marker in a sample by nanoscale flow cytometry.

In one aspect, there is provided a method of enumerating markers on microparticles by nanoscale flow cytometry, the method comprising calibrating a flow cytometer with a control sample comprising enveloped virus particles as described herein, and, following calibration, enumerating markers on microparticles in a sample by nanoscale flow cytometry. In one embodiment, the enumeration may be accomplished by linear regression.

In one aspect, there if provided a method of enumerating markers on viral particles or extracellular vesicles, the method comprising calibrating a flow cytometer with a control sample comprising enveloped virus particles as described herein, and enumerating markers on viral particles or extracellular vesicles in a sample by nanoscale flow cytometry. In one embodiment, the enumeration may be accomplished by linear regression. In some embodiments, the markers on the control sample and viral particles or extracellular vesicles are fluorescently labelled. They may be labelled with the same fluorophore. In some embodiments, number of markers on the control sample may be determined by prior comparison to an MEF bead having a defined number of florescent particles, e.g., as established by the NIST.

In the above, in some embodiments, the sample may be from a subject. The subject may be mammalian in some embodiments. The subject may be human in some embodiments.

In some embodiments, detection of viral particles or extracellular vesicles comprising the at least one marker may be indicative of a biological parameter, such as a disease parameter. In some embodiments, a number of markers above or below a threshold value may be indicative of a biological parameters, such as a disease parameter. The biological parameter may be presence or absence of a disease. For example, the biological parameter may be cancer, pre-cancer, disease staging, disease severity, infection, inflammation, apoptosis, a metabolic state, treatment response, or affected tissue or cell type identity.

In some embodiments, the nanoscale flow cytometry is for measurement of particles less than 250 nm in size. In one embodiment, the nanoscale flow cytometry is for measurement of particles less than 200 nm in size. In one embodiment, the nanoscale flow cytometry is for measurement of particles less than 120 nm in size. In one embodiment, the nanoscale flow cytometry is for measurement of particles less than 100 nm in size. In one embodiment, the nanoscale flow cytometry is for measurement of particles less than 50 nm in size.

### Size/Calibration Ladder

In one aspect, there is provided a size standard or calibration ladder for nanoscale flow cytometry, comprising a plurality of types of enveloped virus particles, each of the enveloped virus particles being fluorescent, wherein each of the types is a different size.

A “ladder” will be understood of a mixture of virus particles types having different sizes. It will be understood that the “type” refers to population of virus particles characterized by a particular size. This size would be understood as different median size, and some heterogeneity is to be expected within a single population of the same type of viral particles. Size can be determined, e.g. by cryro-EM. Each type of virus particle in the ladder in the ladder may comprise the same fluorophore, be it a dye or a fluorescent protein. Alternatively, each type of virus particle in the ladder may comprise a different fluorophore. When different, the fluorophores, for example may absorb and/or emit at different wavelengths. With a given ladder, one or more types of virus particles may comprises a dye and one or more types may comprise fluorescent proteins. Alternatively, depending on requirements, the ladders may comprise virus particle types comprising only dyes or only fluorescent proteins.

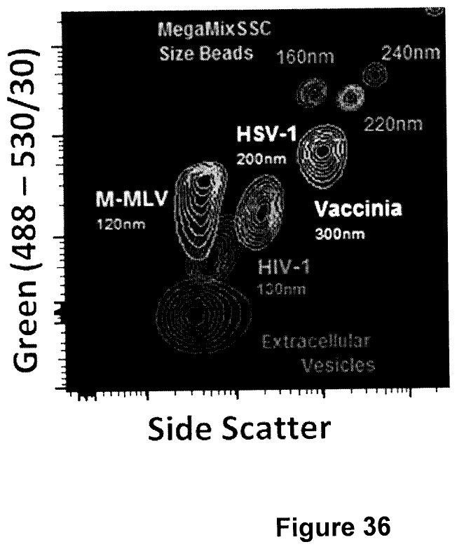

The enveloped virus particles of a ladder may comprise MLV, ALV, HIV, HSV, Influenza, and/or VSV particles. In one embodiment, the ladder may comprise gammaretrovirus particles. In one embodiment, the gammaretrovirus particles are CAS-BR-E, MLV 1313 (Amphotropic MLV), Pmv11 (Polytropic MLV), Xmx15 (Xenotropic MLV), FrMLV (Friend MLV), M-MLV (Moloney MLV), DG-75, AKV MLV (AKV MLV), SL3-3 MLV, E-MLV (Esotropic MLV), Rauscher MLV, Mus Dunni endogenous virus, Abelson MLV, XMRV, Porcine endogenous type C, Gibbon leukemia virus, Baboon endogenous virus strain M7, Feline leukemia virus, Koala retrovirus, or Wooly monkey virus. In one embodiment, the enveloped virus particles comprise M-MLV particles. In one embodiment, the ladder may comprise the virus particles listed in FIG. 36. In one embodiment, these virus particles may be labelled with eGFP.

In one embodiment, the virus particles are as defined above. In one embodiment, the particle comprise particles encoded by the recombinant nucleic acid as described above.

### Production of Controls

In one aspect, there is provided a method of producing fluorescent enveloped virus particles comprising at least one selected marker, the method comprising: infecting a host cell expressing the at least one selected marker with enveloped virus particles, and recovering enveloped virus particles produced by the infected host cell, wherein the recovered enveloped virus particles comprise the at least one selected marker, and wherein the recovered enveloped virus particles are fluorescent.

In this context, the term “expressing” is not intended to limit the marker to an endogenous marker. The marker may be endogenous. However, the cell may also be expressing and exogenous marker, e.g. stably or transiently. In some embodiments, the method comprises a step of manipulating the host cell to express the at least one marker. For example, the host cell may be transformed, infected, or modified (e.g. by CRISPR), as non-limiting examples.

In one embodiment, the method further comprises labelling the recovered enveloped virus particles with a fluorescent dye.

In one embodiment, the recovered enveloped virus particles each comprise one or more fluorescent protein. In one embodiment, the fluorescent protein is enhanced green fluorescent protein (eGFP). In one embodiment, the enveloped virus particles each comprise viral envelope proteins, each labelled with the fluorescent protein. The fluorescent protein may be encoded by a modified viral genome. However, the virus may also be pseudotyped with the fluorescent protein in some embodiments.

In one embodiment, the viral envelope proteins are M-MLV envelope proteins.

In one embodiment, the enveloped virus particles are pseudotyped with the viral envelope proteins.

In one embodiment, the viral envelope proteins labelled with fluorescent protein are as encoded by SEQ ID NO: 1.

In one embodiment, the pseudotyping is accomplished with a vector comprising SEQ ID NO: 2.

In one embodiment, the enveloped virus particles are pseudotyped to be non-infectious to humans.

In one embodiment, the enveloped virus particles are encoded by a nucleic acid comprising at least one sequence modification that reduces or abrogates expression of the endogenous viral envelope proteins.

In one embodiment, the enveloped virus particles comprise MLV, ALV, HIV, HSV, Influenza, and/or VSV particles.

In one embodiment, the enveloped virus particles comprise gammaretrovirus particles. In one embodiment, the gammaretrovirus is CAS-BR-E, MLV 1313 (Amphotropic MLV), Pmv11 (Polytropic MLV), Xmx15 (Xenotropic MLV), FrMLV (Friend MLV), M-MLV (Moloney MLV), DG-75, AKV MLV (AKV MLV), SL3-3 MLV, E-MLV (Ecotropic MLV), Rauscher MLV, Mus Dunni endogenous virus, Abelson MLV, XMRV, Porcine endogenous type C, Gibbon leukemia virus, Baboon endogenous virus strain M7, Feline leukemia virus, Koala retrovirus, or Wooly monkey virus.

In one embodiment, the enveloped virus particles comprise M-MLV particles.

In one embodiment, the enveloped virus particles are encoded by the recombinant nucleic acid described above or the vector as described above.

In one embodiment, the at least one selected marker is endogenous to the host cell.

In one embodiment, the at least one selected marker is exogenous to the host cell.

In one embodiment, the at least one selected marker may be characteristic of a particular cell type or cellular state. In one embodiment, the at least one selected marker may be operationally specific to a particular cell type or cellular state. In one embodiment, the at least one selected marker may be unique to a particular cell type or cellular state.

In one embodiment, the at least one marker expressed by the host cell comprises a plurality of markers of a selected profile, and the recovered enveloped virus particles comprise the plurality of markers of the selected profile.

In one embodiment, the selected profile may be characteristic of a particular cell type or cellular state. In one embodiment, the selected profile may be operationally specific to a particular cell type or cellular state. In one embodiment, the selected profile may be unique to a particular cell type or cellular state.

In some embodiments, the at least one marker may be modified to increase incorporation into the viral envelope upon egress, in some embodiments. For example, the at least one marker may be a recombinant protein comprising the transmembrane (TM) domain of the native viral envelope glycoprotein. In other embodiments, the marker may be modified with a membrane signal peptide In order to direct the insertion of a protein into a membrane and increase incorporation into viral particles upon egress.

In some embodiments, the at least one selected marker(s) may be characteristic of disease cells. For example, marker may be indicative of infection with a pathogen, a state of inflammation, an altered metabolic state, apoptosis, pre-cancer, or cancer. The marker(s) may indicate the presence, stage, or severity of disease; or may provide information about the affected cell type (e.g., the location of an infection).

In one embodiment, the at least one selected marker is a recombinant protein that is modified to promote incorporation of the marker into the recovered enveloped virus particles. In one embodiment, the recombinant protein comprises a transmembrane (TM) domain of a native viral envelope glycoprotein. In one embodiment, the recombinant protein comprises a membrane signal peptide.

In some embodiments, there are provided recovered enveloped virus particles produced by the forgoing method.

In one aspect, there a is a method providing a method of producing a control for the detection of extracellular vesicles (EVs) or virus particles comprising at least one marker, the method comprising carrying out the above method, wherein the at least one selected marker is determined based on the marker of the extracellular vesicles (EVs) or enveloped virus particles to be detected. In one embodiment, the at least one marker comprises markers of a selected profile, which are determined based on a marker profile of the extracellular vesicles (EVs) or enveloped virus particles to be detected.

In some embodiments, method comprise a step of profiling a disease sample to determine the at least one marker or marker profile.

In some embodiments, there is provided a control produced by the forgoing method. The control may be compared to MESF beads to determine the number of markers per control particle, as described here.

### EXAMPLE 1

### Introduction

Retroviruses, such as the human immunodeficiency virus (HIV), are enveloped RNA viruses that range between 90-150 nm in diameter, depending on the species. When nascent virions egress from the surface of infected cells, they bear contents of the parental cell, most notably the cellular membrane to form the viral envelope. As such, by profiling and characterizing the abundance of antigens at the surface of viruses it is possible to identify the infected parental cell type. This is of particular interest for the field of HIV research, where a significant hurdle to curing the disease remains the clearance of elusive infected cell reservoirs. Infected reservoir cells are likely distributed throughout the infected host and are present in various tissues. The very small number of these cells in patients treated with combinational antiretroviral therapy (cART), make their identification notoriously difficult.

Nanoscale flow cytometry (NFC), also called flow virometry, is a new and powerful tool in the field of virology that enables the phenotypic analysis of markers at the surface of individual virions²⁸⁻³⁶. Virus populations can now be profiled and sorted in multi-parameter analyses, much in the same way as cells^{29,31,34,36,37}. However, in first attempts to analyze murine leukemia virus (MLV) particles by NFC, numerous challenges were encountered. These included high background noise, poor resolution and sensitivity, contaminations by extracellular vesicles (EVs), and particle counts that often did not reflect the titer of the input virus. EV is a broad term that describes a particle with a membrane bilayer released from cells; these can include exosomes, microvesicles, and apoptotic vesicles^{38,39}. Small EVs, that are in the size range of retroviruses, are biochemically and biophysically similar to retroviruses in terms of their refractive indices and buoyant densities, and constitute a major contaminant of virus preparations.

Part of the challenges in analyzing viruses and other nanoparticles, such as (EVs), by NFC is the lack of standardized instruments, acquisition settings, and labeling procedures. To complicate matters, instruments of different make will have different optics, fluidics, electronics, detectors, and software. Even using the same instrument model, variations in sensitivity and resolution are common given that the instruments are operating at the threshold of their physical limits of detection. This is often much lower than the published manufacturer's specifications. Most commercial conventional flow cytometers claim that 300 nm is the minimum size limit for the detection of nanoparticles by scattered light. Despite these caveats, several groups are currently using conventional flow cytometers designed for cells to analyze nanoparticles in the 90-150 nm size range^{28-37,40-49}. Common instrument hardware requirements for small particle detection include greater laser power and the use of photomultiplier tubes (PMTs) instead of photodiodes for forward scatter detection. However, there is currently no consensus in the field as to the minimum laser power and voltages required for the detection and resolution of nanoparticles, or whether it more favourable to trigger on fluorescence or scattered light. While conventional flow cytometers clearly have the capacity to detect nanoparticles to varying degrees of sensitivity, standardization of instrument settings and sample acquisition procedures is necessary for cross-laboratory data validation and reproducibility.

Here, a systematic approach was undertaken to analyze viruses by NFC using a BD LSR Fortessa flow cytometer. The model virus for our study is Moloney MLV (M-MLV), as it is a well-studied and characterized retrovirus that is non-infectious for humans. Optimal settings for laser power and voltage were identified to provide maximum particle resolution and sensitivity, as well as sample dilutions and flow rates to minimize coincidence. Data acquired using either fluorescence or side scattered light (SSC) as a trigger for the detection was then compared. Finally, various dyes and staining methods were compared in an attempt to discriminate MLVs from the EV contaminants.

A mutant murine leukemia virus (MLV) has been engineered. A modified

Moloney murine leukemia virus (M-MLV) (derived from NCBI: NC_001501) has been modified or mutated so as to contain a CTG-to-CTA mutation that abolishes the alternative initiation of translation site of the viral glyco-Gag (gPr80) protein. The virus also has the eGFP coding sequence inserted in the proline-rich region of the env gene. The eGFP reporter is therefore present on the external surface of the virus and is antibody accessible. This CTG viral mutant expresses higher green fluorescence than the parental virus and displays a more homogenous cluster of virions. M-MLV is approximately 120 nm in size, as determined by electron cryo-microscopy.

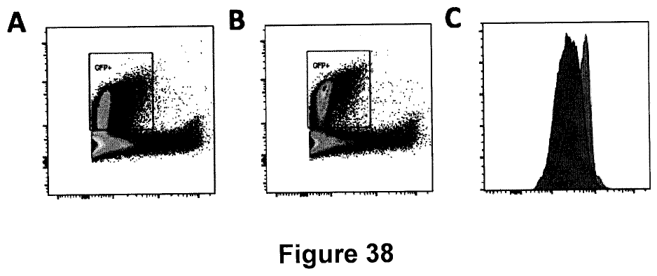

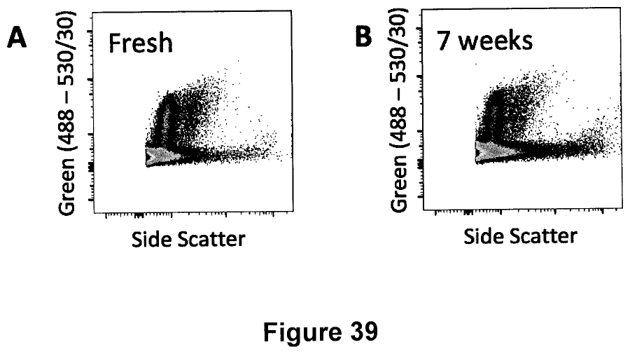

This MLV is fluorescent (MLVeGFP) and released from infected cells as a tight cluster in the 120 nm size range (FIGS. 36 and 37). This mutant MLV called MLVeGFP(CTG) is more fluorescent and more homogeneous in size and fluorescence than the wild type virus (FIG. 38).

The use of MLVeGFP(CTG) to optimize flow cytometer instrument settings. The distinct virus population allows for easy optimization of voltages and laser power for NFC.

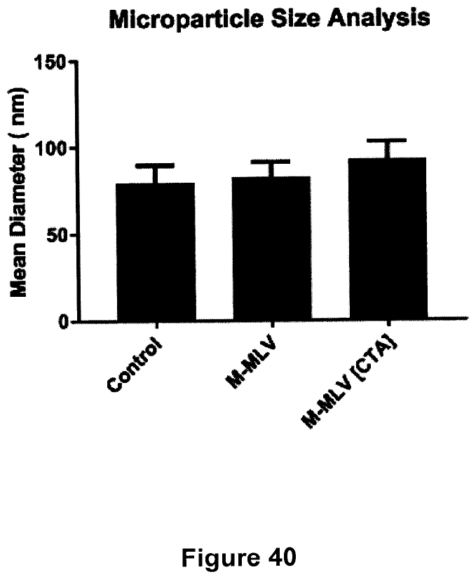

The MLVeGFP(CTG) produced share similar biophysical properties (e.g., buoyant density, refractive index) as extracellular vesicles (EVs) of similar size, most notably exosomes (FIGS. 40).

MLVeGFP(CTG) viruses scatter light (as detected by SSC) similarly to EVs of the same size due to the very similar refractive indices of these particles.

### Methods

### Flow Cytometer Features and Specifications

SORP BD LSRFortessa for small particle detection with a PMT for forward scatter detection. Specifications for laser wavelengths and power are as follows: 405 nm-50 mW, 488 nm-300 mW, 561 nm-50 mW, and 640 nm-40 mW. Acquisition was done with BD FACSDiva version 8.0.1. BD Coherent Connection software was used for laser power adjustments. Samples, unless otherwise indicated, were acquired on low (at 5 turns on the fine adjustment knob), which equated to a measured flow rate of 20 μl/min. This instrument is run with a BD FACSFlow Supply System (FFSS) for day to day acquisition of cells, however for small particle detection, a dedicated steel sheath tank with a 0.1 μm inline filter was used along with a separate waste tank. This was done because we found the FFSS contributed to excess fluctuations in instrument background noise. Surfactant-free, ultra-filtered, low particle count sheath fluid was used for acquisition (Clearflow Sheath Fluid-Leinco). CS&T was run using the dedicated tank to obtain appropriate laser delays for use with the tank, since there is a difference in pressure between the FFSS and the steel tank. Instrument cleaning procedure prior to acquisition: 10 mins distilled water, 30 min FACSClean (BD Biosciences), 10 min distilled water, 60 min 10% Decon™ Contrad™ 70 Liquid Detergent (Thermo Fisher Scientific), and 10 min distilled water.

### Fluorescence Index Calculation

The fluorescence index was calculated as the difference of the median fluorescence intensity of the positive and negative population divided by the standard of deviation of the negative population. Fluorescence index=(MFIpos−MFIneg)/SDneg.

### Data Acquisition and Analysis

Flow cytometry data was displayed in height for all figures. For small particle analysis, height is the preferred parameter over area. Area is the integrated value of an electronic pulse based on the height and width (time of flight). However, since the particles of interest are very small, the width or time of flight measurements become less precise. This leaves height as the intensity of the signal as the most accurate parameter for analysis of submicron-sized particles.

In the instrument platform, side-scatter was chosen over forward-scattered light detection for the approximation of particle sizes. As approximated by Mie Scatter Theory, the angle of light scatter from a particle in the 100-200 nm size range is such that more light is captured at the side-scatter angle rather than forward, whereas with a cell-sized particle (10 μm) the opposite is true^{45,61}. It was found that, despite having a PMT for FSC detection, resolution in SSC was still superior for the particles of interest. Flow cytometry data was analysed using Flowjo VX (FLOWJO, LLC). GraphPad Prism v6 was used for the generation of graphs (GraphPad Software).

### Fluorescent Polystyrene Beads

Megamix-Plus SSC fluorescently labeled beads (Biocytex, Marseille, France #7803) with 160 nm, 200 nm, 240 nm, and 500 nm size populations was used. The 500 nm bead population was off scale when run at settings optimized for MLVeGFP resolution.

### Cells, Plasmids and Viruses

Human embryonic kidney (HEK) 293T and mouse embryonic fibroblast NIH 3T3 cells were cultured in phenol-red free DMEM High Glucose Medium (Wisent, St Bruno, Canada), supplemented with 10% Fetal Bovine Serum (FBS, Gibco by Thermo Fischer Scientific, Waltham, Mass.), 100 U/mL penicillin and 100 μg/mL streptomycin (Wisent, St Bruno, Canada). This media will be referred to as complete media. Propagation was continued at 37° C. in a 5% CO₂ incubator.

### Transfections

Moloney-MLV, referred to as MLVeGFP throughout this study, was produced from the pMOV-eGFP expression plasmid⁶²⁻⁶⁴. The eGFP is located on the exterior surface of the virus, within the proline-rich region of the viral envelope glycoprotein, and does not alter infectivity nor ecotropic receptor specificity⁶². The plasmid eGFP-C3 (Clontech, Mountain View, Calif.) was used for cytoplasmic eGFP expression. For the construction of an Env-eGFP plasmid, Env-eGFP of MLVeGFP was amplified by PCR using the following primers: GCTAGCGCCGCCACCATGGCGCGTTCAAC GCTCTCAAAACC (forward) and CTCGAGCTATGGCTCGTACTCTATAGGCTTCAGCTG GTG (reverse). The amplification product was then inserted between the NheI and XhoI restriction sites of the expression vector pcDNA 3.1 (−) downstream of the CMV promoter.

For virus or EV production, 293T cells were transfected with MLVeGFP, Env-eGFP or eGFP-C3. 24 h before transfection, 293T cells were seeded at a density of 1.25×10⁵ cells/well in a 24-well plate in complete media. For each well, a total of 500 ng of DNA was transfected using GeneJuice (Novagen, EMD Millipore, Billerica, Mass.) according to manufacturer's instructions. After 36 h, the media was changed to 0.1 μm-filtered, serum and phenol red-free DMEM (Wisent), to allow for virus or EV production with minimal contaminants. After 3 h, the cell supernatant was harvested and cleared through a 0.45pm acrodisc syringe filter with SuPor (PES) membrane (Pall Corporation, Port Washington, N.Y., cat. #PN4614) unless otherwise specified. For analysis of the effects of microfiltration on viral sample, supernatants were filtered through 0.1 μm (cat. #PN4612), 0.2 μm (cat. #PN4612) or 0.45 μm filters (all Pall Corporation). Samples produced from transfections were diluted 1:10 prior to analysis by NFC.

### Cell Infections

To generate cells chronically infected with MLVeGFP, NIH 3T3 cells were infected with MLVeGFP at a high multiplicity of infection (MOI). In short, 10 mL of MLVeGFP containing cell supernatant was produced by transfection for 72 h. The supernatant was cleared through a 0.45 μm filter and was ultra-centrifuged at 100,000×g for 3 h in a 70Ti rotor at 4° C. The entire viral pellet was used to infect NIH 3T3. Uninfected control cells or chronically infected NIH 3T3 cells were seeded at a density of 5×105 cells/well in 6-well plates. After 18 h, cells were washed 3 times with 0.1 μm filtered PBS and incubated in 0.1 μm filtered, serum and phenol red-free DMEM for 3 h.

### Vaccinia Stock Preparations

VVDD-mCherry stocks were obtained from John C. Bell (OHRI). Briefly, virus stocks were produced by infecting HeLa cells at a low MOI (0.01). Infected cells were lysed by repeat freeze-thaw cycles (−80° C./37° C.), cell debris was removed by centrifugation, and virus was clarified through a sucrose cushion at 20,700 RCF with a JS-13.1 rotor for 80 min at 4° C.^{32,65}.

### Nanoparticle Tracking Analysis

Nanoparticle tracking analysis (NTA) was carried out using the ZetaView PMX110 Multiple Parameter Particle Tracking Analyzer (Particle Metrix, Meerbusch, Germany) in size mode using ZetaView software version 8.02.28. Samples were diluted in PBS to ˜10⁷ particles/ml. The system was calibrated using 105 and 500 nm polystyrene beads and then videos were recorded and analyzed at all 11 camera positions with a 2 second video length, a camera frame rate of 30 fps and a temperature of 21° C. Analysis was performed using Particle Explorer version 1.6.9 (Particle Metrix). Analysis parameters were: segmentation-fixed, centroid estimation-blob, drift compensation-auto, log detection threshold-0.0175, max particle size-1000, min particle size-6.0, segment threshold-18. Results are displayed as the percentage of particles within 25 nm segments and as mean particle size.

### Direct Labeling with Lipid Dyes

Dyes were added directly to undiluted control or MLVeGFP containing supernatant at optimized concentrations. The dye-labeled control or viral supernatants were diluted 1:10 and 1:100 in 0.1 μm filtered PBS, respectively. These were then filtered with a 0.45 μm pore-size syringe filter prior to acquisition on the cytometer. DiD and Dil solutions were used at 10 μM while DHPE-Rhodamine, FM 4-64X and BODIPY TR Ceramide were used at 10 μg/mL (all Thermo Fisher Scientific).

### Indirect Labeling with Lipid Dyes

Uninfected and infected NIH 3T3s were cultured overnight with dye. The following day, cells were washed 3 times with 0.1 μm-filtered PBS to remove excess dye. Following washing, the cells were placed back in the 37° C. incubator with 0.1 μm filtered, serum and phenol red-free media. After 3 h, supernatant was collected, 0.45 μm-filtered (unless otherwise indicated), and analyzed by NFC. As before, control or viral supernatants were diluted 1:10 and 1:100 in 0.1 μm filtered-PBS, respectively. DiD and Dil solutions were used at 25 μM, DHPE-Rhodamine and FM 4-64X were used at 25 μg/mL, and BODIPY TR Ceramide was used at 12.5 μg/mL. DiD and Dil were dissolved in ethanol, while DHPE-Rhodamine, FM 4-64X and BODIPY TR Ceramide were dissolved in methanol. Titrations were performed for both the direct and indirect labeling methods, optimal concentrations were chosen (data not shown).

### Nucleic Acid Labeling

The protocol for nucleic acid labeling with SYBR Green was adapted from Brussard et al.^{26,29}. Briefly, supernatants were fixed in 2% methanol-free paraformaldeyde (PFA) solution (Thermo Fisher Scientific, cat. #28906). Virus samples were stained with 1× SYBR Green I (DNA) or SYBR Green II (RNA) at 80° C. for 10 minutes. For dual labeling of

MLVeGFP, lipophilic dye was loaded onto the virus by indirect labeling prior to nucleic acid staining. For dual labeling of VV, virus particles were labeled using the direct method post SYBR Green I staining. Samples were diluted and 0.45 μm-filtered after staining for analysis by NFC.

### Fluorescence Microscopy

Uninfected NIH3T3 cells were labeled as described above for the indirect staining method, but scaled down to fit 35 mm dishes (Ibidi, Fitchburg, Wis.). The Zeiss LSM 880 was used for live imaging confocal microscopy, ImageJ (1.8.0) was used to generate the images.

### Results

### Analysis of Single-Particle Viruses by NFC

For this study, Moloney MLV expressing a chimeric envelope-eGFP surface glycoprotein (MLVeGFP) was used. Moloney MLV is an enveloped virus that is nearly spherical with a mean diameter of 124 nm as measured by cryo-electron microscopy. It is estimated that there are approximately 100 envelope glycoprotein spikes per virion of MLV, which is nearly an order of magnitude more than the 7-14 gp120 spikes found at the surface of HIV-1. It has been shown previously that enveloped viruses can form aggregates when subject to centrifugation and repeat freeze thaw³⁶. Similarly, EVs form aggregates when centrifuged⁶¹. For this reason, and to reduce other contaminants, the virus was produced from chronically infected NIH 3T3 cells cultured in 0.1 μm filtered, serum and phenol red-free media.

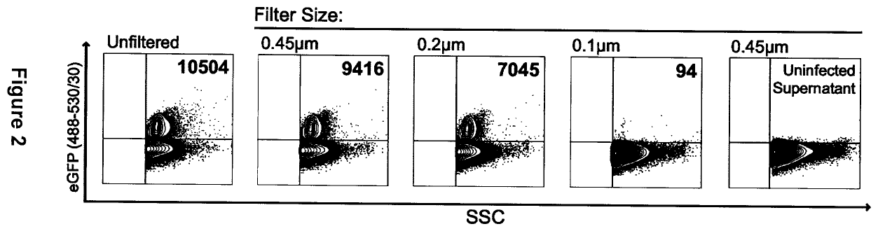

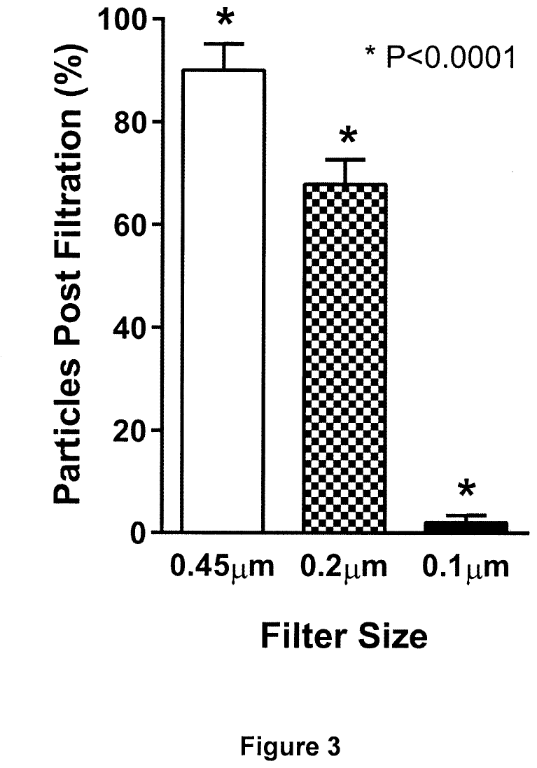

The effect of microfiltration on the virus samples was ascertained (FIGS. 2 to 9). Filtration ensures that cellular debris is removed and that viruses are present in the sample as single particles. Although nearly all the viruses produced will harbour the Env-eGFP protein on their surface, it is unclear at this stage what proportion of all eGFP+ particles constitute virus. Virus-containing supernatants were harvested, directly filtered as described, diluted 100-fold in 0.1 μm-filtered PBS and analyzed by NFC. Filtration through a filter with a 0.45 μm pore size removed approximately 10% of total eGFP+ particles, while filtering through a 0.2 μm cartridge removed 30% of eGFP particles (FIGS. 2 and 3). Passing the sample through a 0.1 μm filter removed nearly all eGFP particles, as expected.

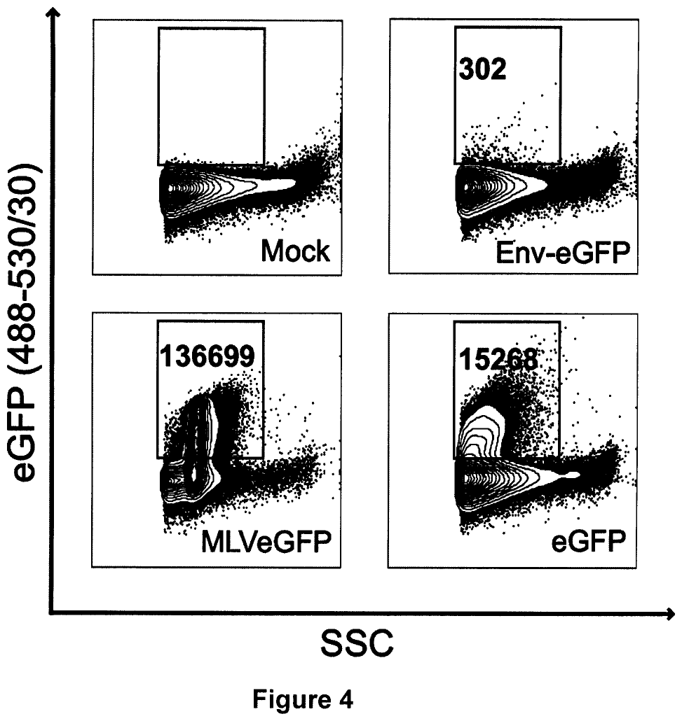

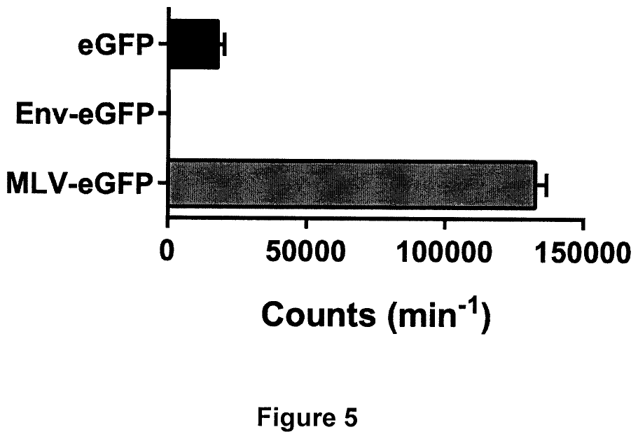

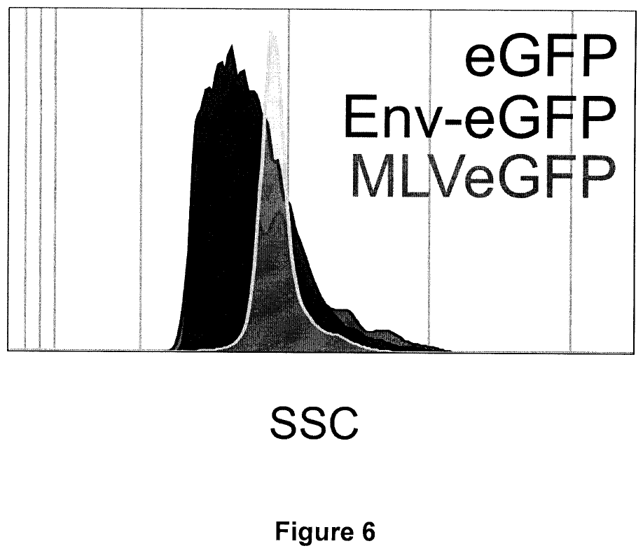

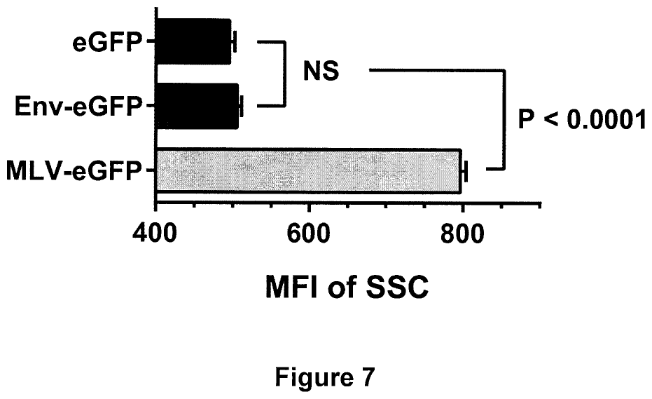

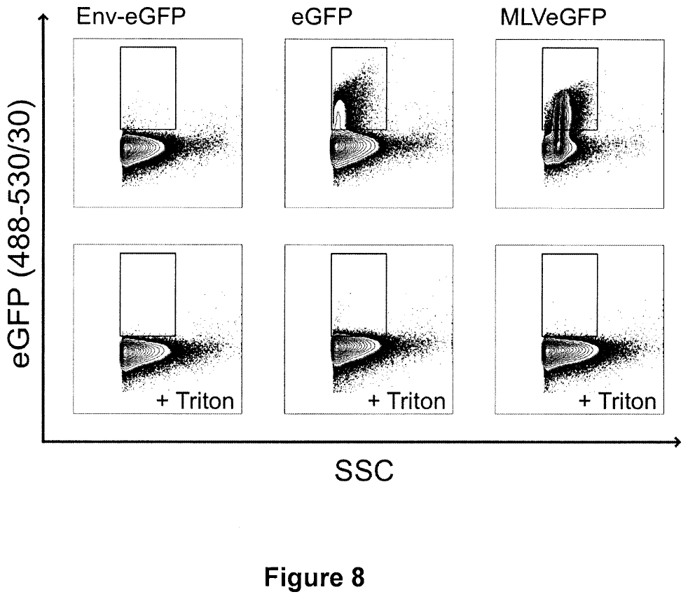

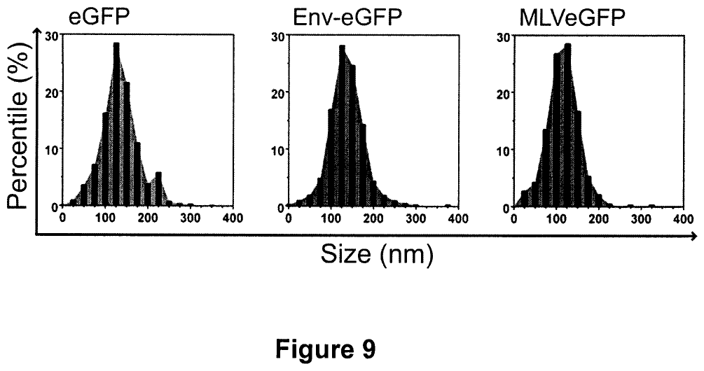

It was then assessed whether eGFP+ particles are indeed mostly viruses. It is well documented that EVs can acquire viral proteins and nucleic acids when they are released from cells. Because MLV viruses are labeled by acquiring Env-eGFP into their envelope, the coding sequence of this protein was cloned in an expression vector and transfected it into 293T cells in order to measure EV uptake of the protein. The virus was also produced by transfection so that experimental conditions would be similar. All the samples were passed through a 0.45 μm filter prior to NFC analysis. It was found that Env-eGFP particles are more abundant in the cell supernatant when the virus is present (FIG. 4). In absence of viral structural proteins that make the capsid, less than 0.25% of Env-eGFP particles were detected compared to MLVeGFP in matched acquisition conditions (FIG. 5). Interestingly, when the cells were transfected with an eGFP expression plasmid, a larger number of particles were detected. This represented about 10% of the total eGFP+ particles of the viral supernatant. eGFP is a cytoplasmic protein, while Env-eGFP is membrane-associated and accumulates at the cell surface. These results indicate that the release pathways of MLVs and EVs are different. All samples were then subject to lysis by 0.05% Triton to confirm that eGFP+ particles were vesicles and viruses, and not protein aggregates (FIG. 8). An interesting observation from the analysis of the NFC data shows that particles released in cells transfected with eGFP or Env-eGFP display noticeably different side scattered light (SSC) values (FIGS. 6 and 7). MLV viruses therefore appear to display higher mean fluorescent intensity (MFI) and a much more homogenous distribution, despite having a similar size distribution profile as measured by Nanoparticle Tracking Analysis (NTA) (FIG. 9). Overall, these data support that most of the eGFP+ particles produced from MLVeGFP infected cells are virus.

### Impact of Thresholding and Sample Dilution on Electronic Aborts and Event Counts

In order to have analyzed eGFP+ particles in the previous section, several NFC parameters had first needed to be optimized which include voltage, laser power, flow rates and sample dilutions. The following sections describe in detail how these settings were determined and also how they affect data acquisition.

Coincidence occurs when two or more particles are interrogated by a laser simultaneously^{43,45,46}. Flow cytometers are specifically designed to create a stream of single cells that are individually analyzed by the instrument. Because nanoparticles are much smaller than cells, several particles can coincidentally be interrogated at the same time if the sample is too concentrated. Additionally, when a particle crosses the path of the laser and is interrogated over a given time frame, photons are scattered by the particle and are then captured by PMTs that convert them into an electromagnetic pulse. If this pulse does not return to a baseline value before it increases again, this will generate an electronic abort event and the data will not be recorded. Therefore, if a very concentrated sample is analyzed, a large number of electronic aborts will be generated and data events will be discarded. This constitutes a major concern in NFC analysis of viruses and EVs. Furthermore, because the signal generated by nanoparticles is very dim, the threshold for detection is set at or near the lower limit. Low threshold values translate into reduced stringency of what is considered an event and this results in increased background noise.

An additional layer of complexity that comes into play that can affect coincidence is thresholding, also called trigger. When thresholding using SSC, the wavelength of the laser interrogating the particle is the same as the one that is captured by the PMTs. Fluorescence, therefore, does not play a role in SSC thresholding. When thresholding off of fluorescence, the particles are interrogated with a laser emitting at the desired fluorochrome excitation wavelength and the PMTs capture only photons with the appropriate emission wavelength. Because there are generally fewer fluorescent particles than total particles in a sample, fluorescent thresholding may provide the benefit of minimizing electronic aborts. However, the compromise is losing information about total particles in the sample, fluorescent and non-fluorescent. Here a systematic comparison of the effects of sample dilutions and flow rates on both SSC and fluorescence thresholding was therefore made.

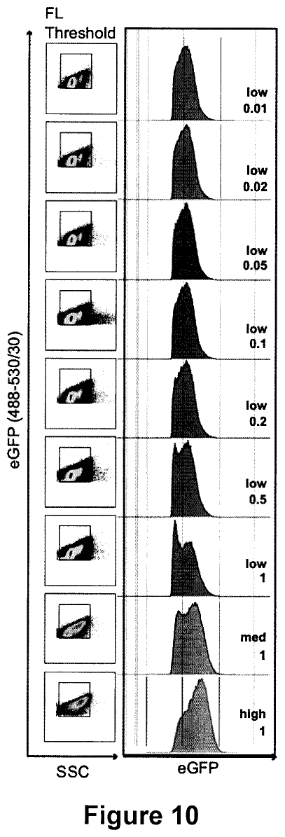

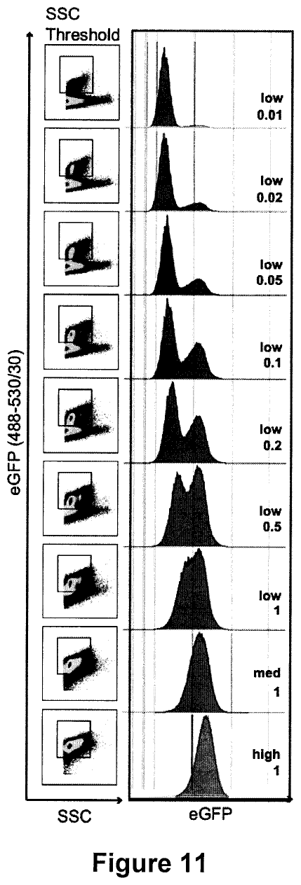

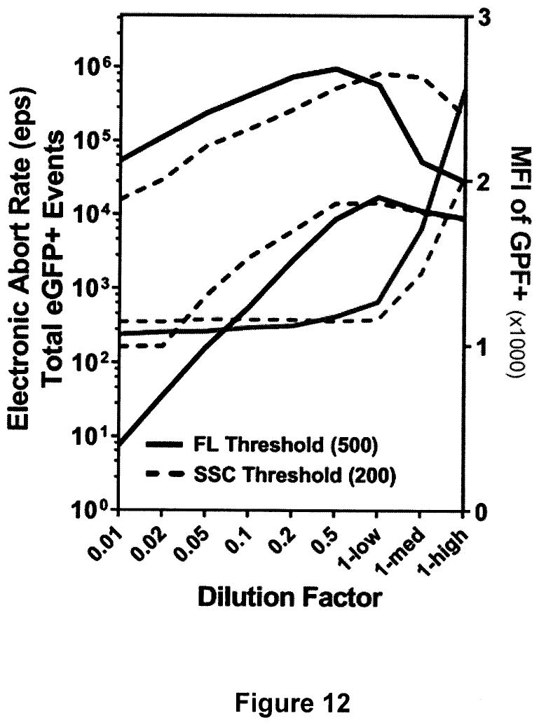

Serial dilutions of 0.45 μm-filtered supernatants containing MLVeGFP produced from chronically infected NIH 3T3 cells were analyzed using the low flow rate setting, and the undiluted sample was analyzed using the low, medium (med) and high settings. The average virus titer in the undiluted filtered supernatant remained constant throughout replicate experiments at approximately 1.5-3×10⁶ transducing units (TU)/mL. The samples were analyzed using fluorescence thresholding (FIG. 10) or SSC thresholding (FIG. 11), and the electronic abort rates were manually recorded. The optimal sample concentration and flow rate is where signal intensities stabilize and events rates linearly correlate with changes in sample concentration^{43,45,46}. Sample acquisition data was plotted to display electronic aborts, total events and fluorescence as a function of sample dilution and increasing flow rates for the undiluted sample (FIG. 12). Total events acquired using SSC

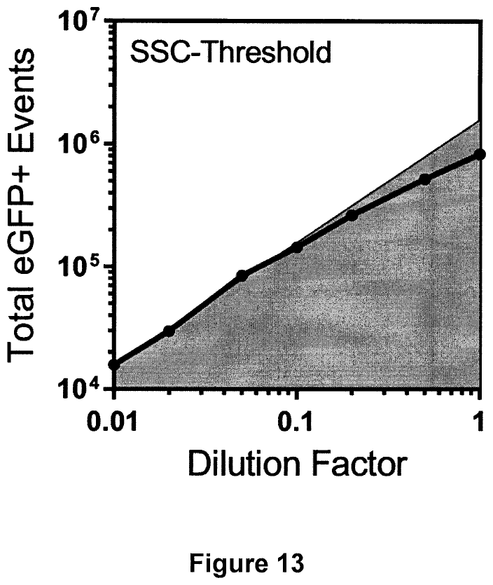

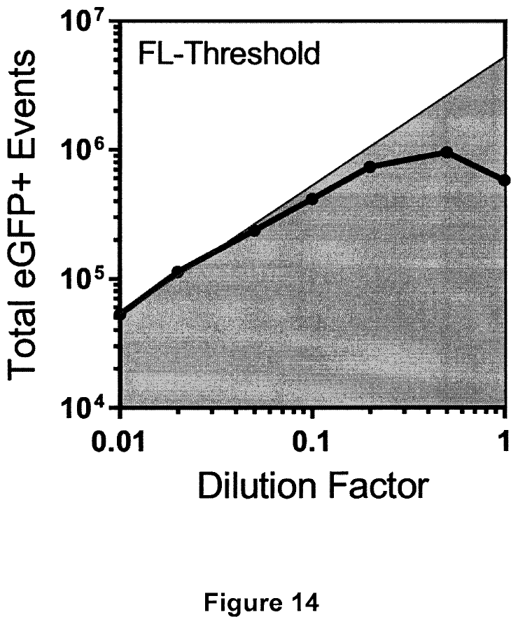

(FIG. 13) and fluorescence (FIG. 14) thresholding were separately plotted against a linear regression curve in order to emphasize the dilutions in the linear range. Linear regression of the predicted number of eGFP+ events (shaded area) was extrapolated using the eGFP+ counts from the most dilute sample for each thresholding condition. Data shows that electronic aborts and coincidence steeply rise as the samples become more concentrated. Consistent with coincidence, an increase in electronic aborts also associates with a saturation of the PMTs, which is exhibited with a rise in mean fluorescence intensity (MFI) of particles (FIG. 12). Total and eGFP particle counts are inversely proportional to the dilution factor of the sample when in the range of 1/100- 1/20 (FIGS. 13 and 14). Surprisingly, contrary to what would be expected, particle counts appear to be slightly lower using SSC compared to fluorescence thresholding by a factor of 3. This could be due to the slightly higher rate of electronic aborts using SSC thresholding even at a dilution of 1/100 (FIG. 12). Taken together, the data indicates that a stable MFI alone does not indicate the sample is running at an optimum event rate. While there is a wide window of dilutions that provide constant MFI values, event count analysis provides a more accurate way of determining optimal flow rates and sample dilutions.

### Effects of Voltage and Laser Power Settings on Particle Detection, Resolution and Electronic Aborts

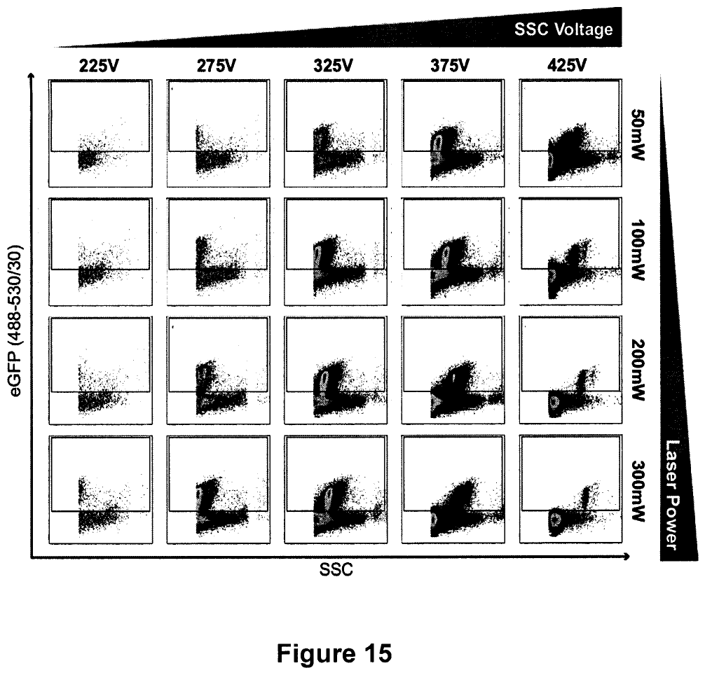

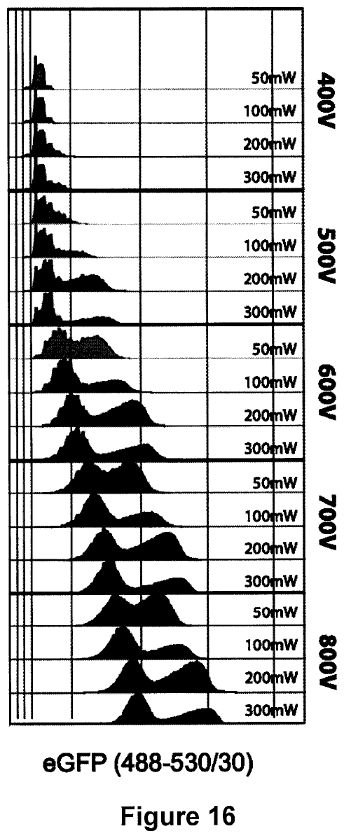

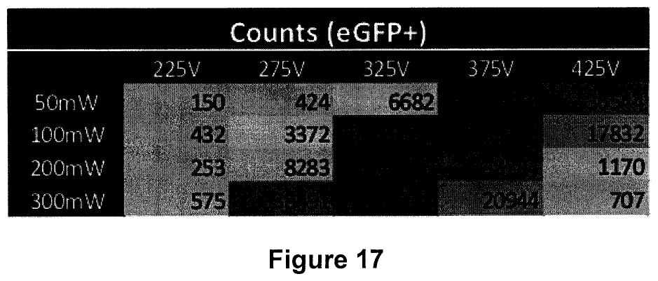

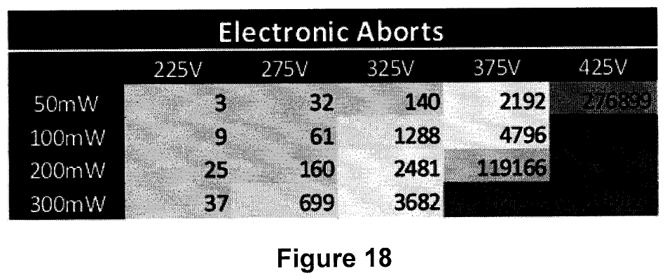

The effect of increasing voltage and laser power on diluted and filtered virus preparations was systematically measured (FIG. 15). This began with the 488 nm laser at full power (300 mW) and systematically increased the SSC voltages in increments of 50V, and reduced laser power all while keeping eGFP MFI constant. In a subsequent acquisition set, green fluorescence (488-530/30) detector voltage was gradually increased in increments of 100V (while keeping the SSC MFI constant) to determine the range of settings at which the eGFP+ virus is detected (FIG. 16). Threshold was set on SSC off the 488 nm laser at the lowest value permitted by our acquisition software, which is 200 relative fluorescence intensity units. It was decided to threshold off of SSC to include detection of all fluorescent and non-fluorescent events. Because acquisition is carried out on the same sample, changes will reflect the effects of the settings. When voltages were increased on SSC, the noise and event rate also increased to a certain limit (FIG. 17), which correlated with increased electronic aborts (FIG. 18). The increased electronic abort rate at higher SSC voltage settings resulted in a loss of the total number of eGFP+ particles acquired.

When comparing data collected for a given SSC voltage setting, increase in laser power resulted in an increase in the separation of SSC signal intensities between the eGFP+ and eGFP-populations (FIG. 15). This is most evident in the voltage range between 325 to 425V for SSC where the eGFP+ population is above threshold on SSC. Furthermore, peak particle counts was reached at a lower voltage as laser power increases (FIG. 17). This again, perfectly tracked with electronic aborts that were at their maximum when laser power and PMT voltages were at their highest settings (FIG. 18).

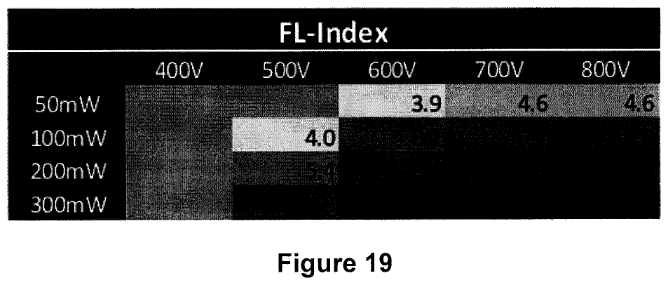

When laser power and voltage on the green fluorescence channel were increased, it was evident that signal intensities of both the eGFP+ and eGFP− populations were also increased (FIG. 16). To determine if increase of laser power resulted in an increase in separation of the positive and negative populations in the green fluorescence channel, a fluorescence index (FL-index) value was calculated. This value is based on the difference in the MFI of the eGFP+ and eGFP− populations divided by the standard deviation of the eGFP− population (FIG. 19). Samples where there was no clear resolution of an eGFP+ population were omitted. When comparing samples acquired at the same voltage, higher laser power provided improved resolution of the fluorescence signal (defined by the separation of the positive population from the negative) as represented by a higher fluorescence index value (FIG. 19). Therefore greater laser power more noticeably improves fluorescent resolution than detection sensitivity, when using SSC as a threshold.

### Direct Staining of Viruses with Fluorescent Dyes

To date, several groups have demonstrated the ability to label EVs and viruses through the use of commercially available dyes. Dyes that target different cellular components, such as proteins, lipids and nucleic acids, have been proven effective to stain EVs and viruses in flow cytometry applications^{30,36,37,46,62}. However it has yet to be demonstrated that these methods can be used to discriminate between viruses and EVs.

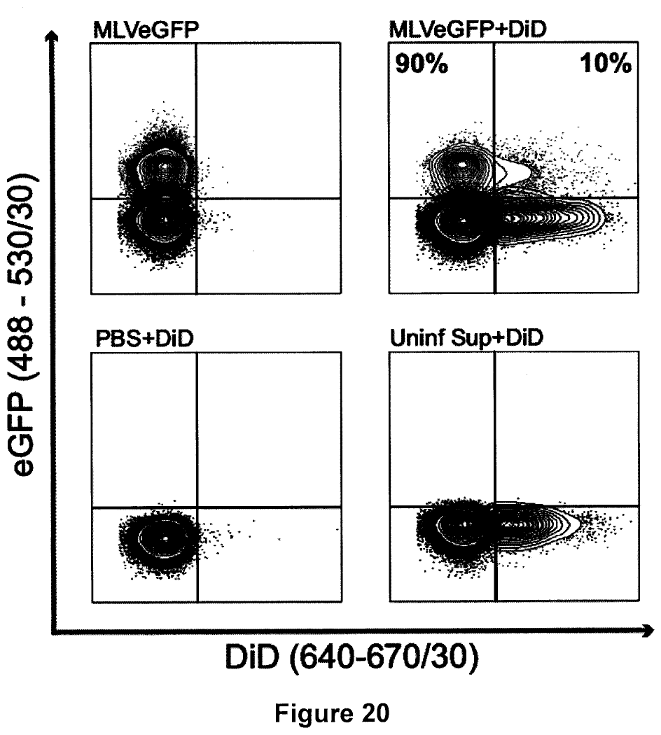

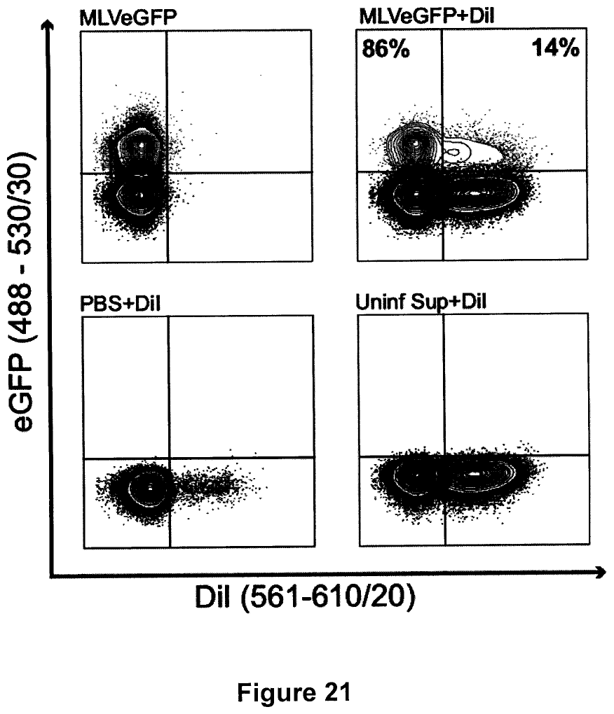

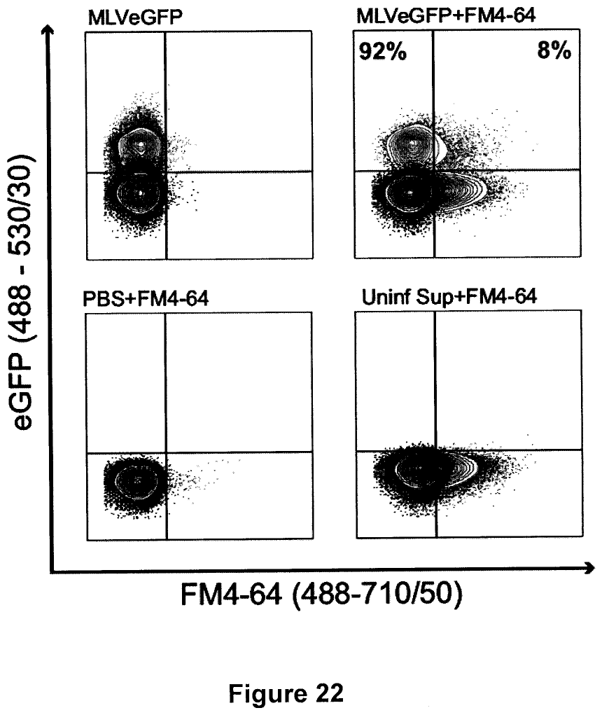

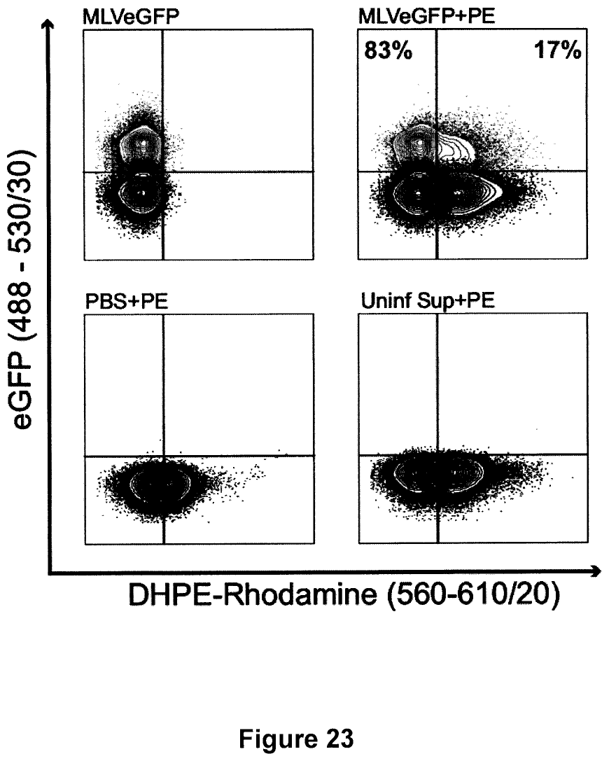

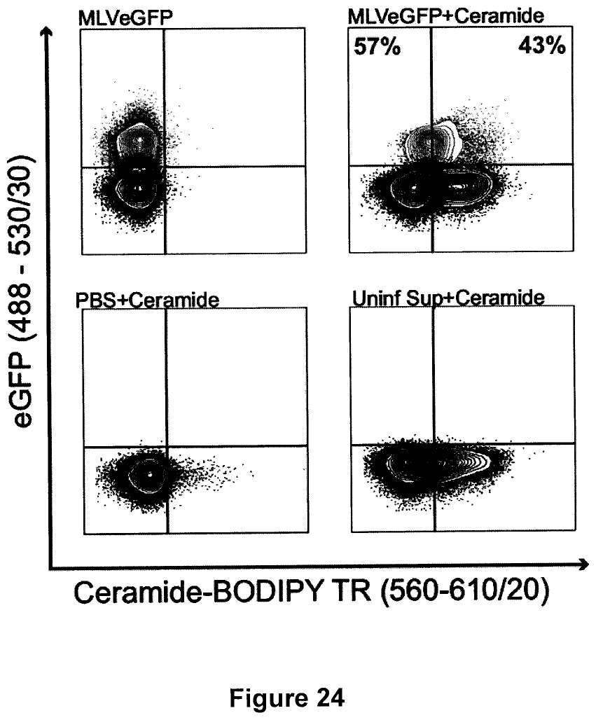

To identify both virus and EV populations in our infected supernatants, fluorescent dyes were used that target cellular membrane components. Dyes were selected over the use of antibodies due to their ability to stain particles based on their biochemical constituents. Dyes are also not subject to some of the limitations of antibodies such as low surface antigen expression, epitope heterogeneity, and particle aggregation³⁶. There is currently a large number of commercially available dyes which target different molecules and cellular components. Using MLVeGFP, and first tested the lipid membrane targeting dyes DiD, Dil, FM4-64, fluorescent sphingolipid (Ceramide-BODIPY TR) and fluorescent phospholipid (DHPE-Lissamine Rhodamine). Several groups have published methods to directly label virus with dye⁶³⁻⁶⁶, however these studies did not account for EV contamination in virus sample preparations or the possibility that the dyes may form micelles. In our system, virus particles will be detectable by Env-eGFP expressed on their surface, and by fluorescence coming from the tested dye. EVs, are largely devoid of Env-eGFP (FIG. 5), and therefore will only emit fluorescence at the wavelength of the dye. For all the dyes tested, the direct sample staining approach did not efficiently stain viral particles, as very few particles were double-positive for eGFP and the dye (FIGS. 20 to 24). Samples were then analyzed by NFC without purification. eGFP+ particles are depicted in green, background noise or non-fluorescent particles in black, dye-labeled particles in color (other than green). The distribution of total dye-labeled and unlabeled eGFP+ particles is indicated in green. Included in each panel: unstained virus (MLVeGFP), stained virus (MLVeGFP+dye), dye alone (PBS+dye), and uninfected supernatant with dye that serves as an EV-only sample (Uninf Sup+dye). Ceramide-BODIPY TR was the only dye that labeled the whole virus population, but this labeled population was not completely resolved from background (FIG. 24). Since excess dye was not washed away in this method, Dye+particles were detected in the PBS+Dye controls, which may reflect the insoluble dye aggregates or micelle formation as evident with other lipophilic dyes⁶². The inability of direct staining to efficiently label MLVeGFP particles lead us to pursue alternative methods for labeling viruses with dye.

### Indirect Staining of Viruses with Fluorescent Dyes

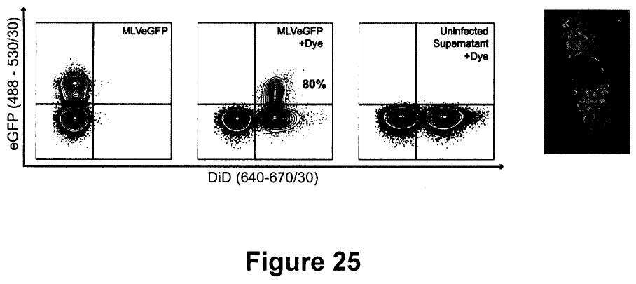

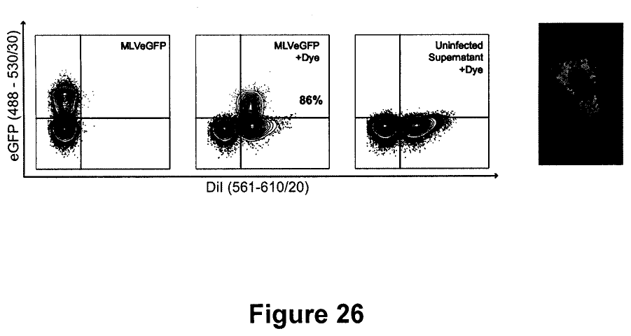

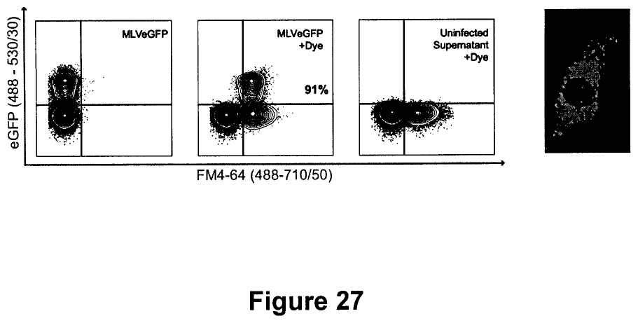

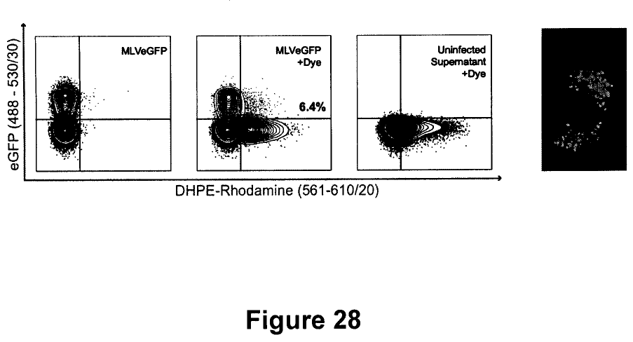

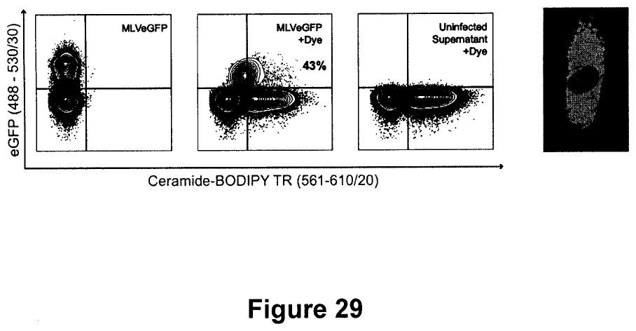

Our next approach was to stain virus infected cells in vitro such that the virus would bud directly from fluorescently labeled lipid membranes. This method has been shown to label extracellular vesicles, but has yet been shown to label virus, as far as is known. The same panel of dyes as above were tested. Staining on cells was confirmed by fluorescence confocal microscopy (FIGS. 25 to 29, right panels). The population of MLVeGFP labeled in vitro with DiD, Dil and FM4-64 was nearly completely resolved from noise by each of these dyes (FIGS. 25 to 27). DHPE did not significantly label MLVeGFP (FIG. 28), while Ceramide appeared to label similarly to the direct staining method, and again was unable to resolve the virus population from the background noise (FIG. 29).

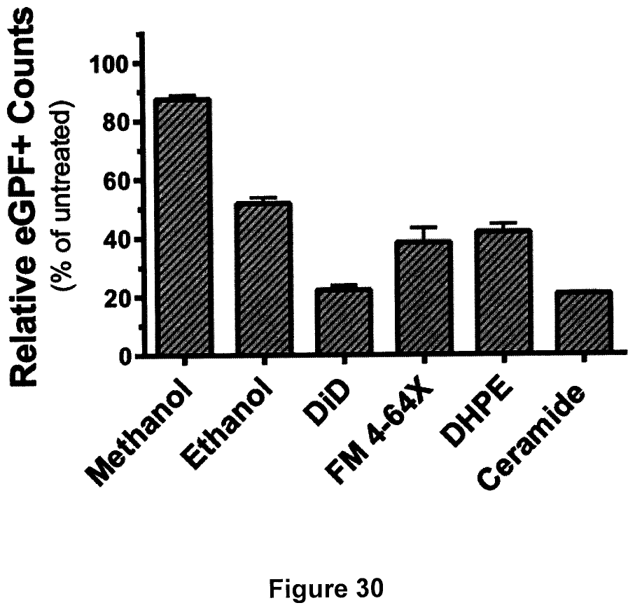

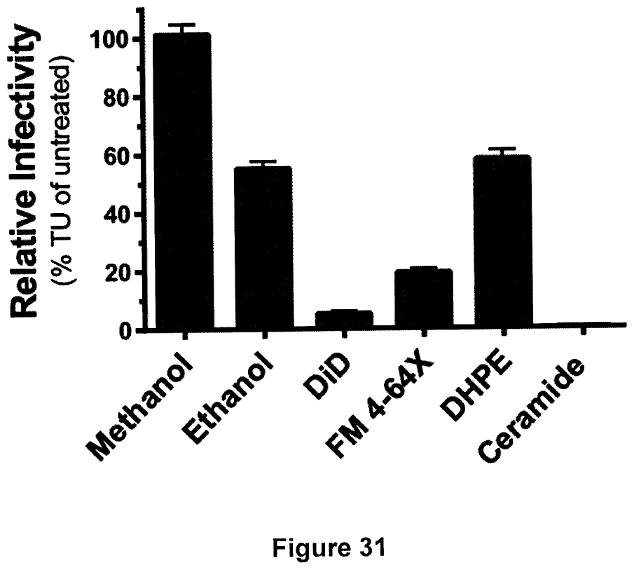

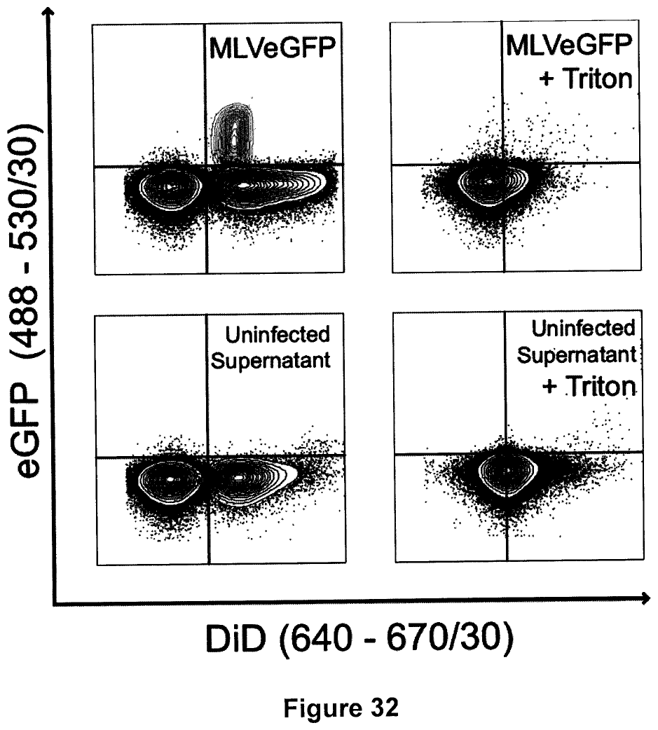

Next it was assessed if stained viruses remained infectious. This could be of use for downstream applications that may investigate virus targeting to certain cells. Dil stained cells were excluded from this assessment as it is chemically analogous to DiD. The effects of solvents were included as controls. The infectivity of viral supernatants produced in DiD and Ceramide labeled cells was abrogated, while Rhodamine-PE and FM4-64 labeled cells produced virus with significantly reduced infectivity (FIG. 30). Although, virus infectivity may be compromised by the dyes, it was also assessed whether treating the infected cells with the dyes affected virus particle release. Indeed, fewer viral particles were released with most of the dyes (FIG. 31), however this did not account for the total loss of infectivity. To confirm that fluorescent particles were indeed viruses and EVs, samples were treated with 0.05% Triton. The EV population was abolished by detergent treatment, as was eGFP fluorescence emitted by the virus (FIG. 32).

### Discriminating Enveloped Viruses From EVs by Staining Nucleic Acids

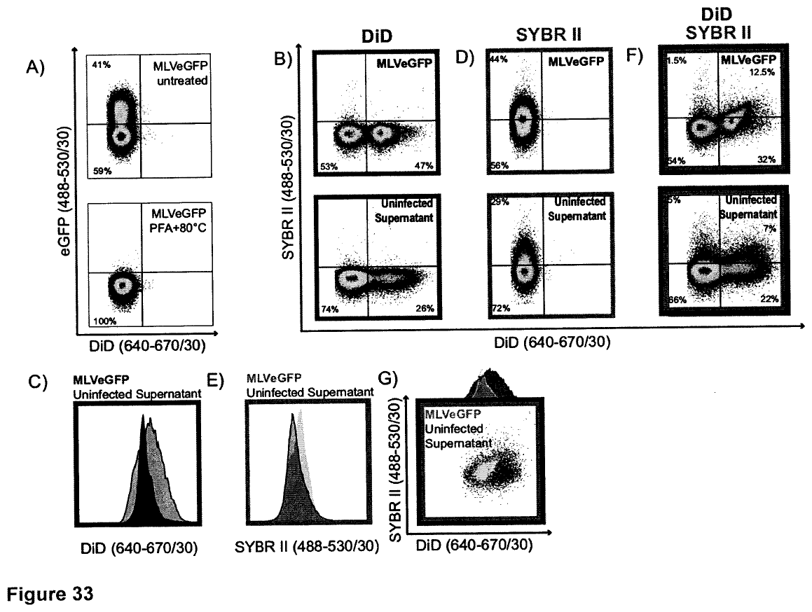

With the indirect staining method, it was clear that some of the dyes were able to resolve the MLVeGFP population. However, EVs were also being labeled by all the dyes tested. It was next attempted to discriminate EVs from viruses based on their nucleic acid content. SYBR Green is a dye that has a strong affinity for nucleic acids, with subtypes that have been developed with higher affinities/quantum yields for RNA (SYBR II) or DNA (SYBR I). Nucleic acid dyes such as SYBR Green have been shown previously to label virus, however in those studies it was unclear whether the dye-labeled virus samples were contaminated with EVs (ref). To discriminate EVs from viruses, MLVeGFP was first labeled with the lipid membrane dye DiD followed by SYBR II, and vaccinia virus (VV) was stained with Dil and then with SYBR I. MLV packages two copies of its 9 Kb single stranded RNA genome (Ref), while the VV harbours a double-stranded DNA genome of about genome 200 Kb (ref). It was predict that the higher genomic content of our viruses should distinguish them from the EV contaminants, which have been reported to carry up to 2 Kb in dsDNA cargo (Confirm amount & ref). While many different types of RNA species have been identified in EVs, relative abundance varies based on cellular origin (ref). Furthermore, their total capacity for packaging nucleic acid remains to be determined. The nucleic acid labeling procedure, as described previously (ref), required fixation of the virus followed by staining at 80° C. This exposure to heat denatured the eGFP on the MLVeGFP and it was no longer fluorescent (FIG. 33, Panel (A)). Fixation with 2% paraformaldehyde (PFA) alone had no significant impact on eGFP fluorescence (data not shown). MLVeGFP and uninfected cell supernatants were labeled alone with DiD (indirect) (FIG. 33, Panels (B) and (C)), or SYBR II (direct) (FIG. 33, Panels (D) and (E)), and dual labeled (FIG. 33, Panels (F) and (6)). When the DiD+ population in the MLVeGFP sample was compared with that of the uninfected supernatant control, the two populations exhibited different MFI despite largely overlapping (FIG. 33, Panel (C)). The same was true for the SYBR II positive and double labeled populations (FIG. 33, Panels (E) and (G)). This indicates that MLVeGFP and EVs contain similar amounts of dye-labeled nucleic acids.

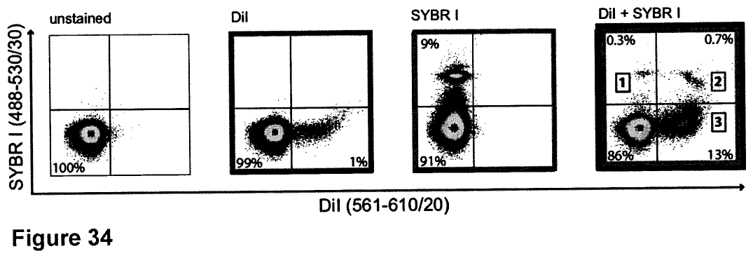

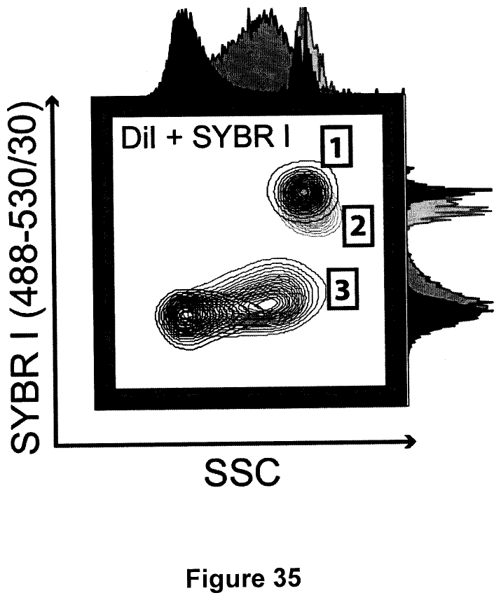

While Dil labeling of VV was unable to distinguish the virus from other cellular vesicles, the use of SYBR I distinctly resolves a defined population (FIG. 34). Dual labeling produced three populations: DiI+SYBR+, Dil−SYBR+, and DiI+SYBR (FIGS. 34 and 35). This indicates that the larger genome size of VV enabled a clear separation of the virus from EVs.

### Ladders

The fluorescent labeling method of the MLVs can be used to label or pseudotype other enveloped viruses (e.g., ALV, HIV, HSV, Influenza, and VSV) of slightly different sizes, and make it therefore possible to construct a molecular size ladder in a narrow 90-200 nm size range (FIGS. 36 and 37). More specifically, the sequence of MLV eGFP-Env was cloned into a retroviral transduction vector and generated stably transduced 293T cells. By infecting these cells with various enveloped viruses species (e.g., ALV, HIV, HSV, Influenza, and VSV) that have slightly different sizes, we are able to pseudotype these viruses with eGFP-Env, thereby labeling the viruses similarly to the MLVeGFP(CTG) virus. Viruses produced are highly fluorescent and can be used together to form a molecular size ladder in the lower range of flow cytometer detection limits (FIG. 36).

### Discussion and Conclusions

Careful optimization of instrument settings is often underappreciated to obtain the best performance from a flow cytometer. In conventional flow cytometers, this optimization is standardized with beads as a reference material. In BD instruments the one used herein, this is achieved with the automated BD CS&T program and beads, however CS&T target values are optimized for cells. Therefore voltages and laser settings for the analysis of nanoscale particles need to be optimized manually with relevant reference materials, especially since the particles of interest are at the limit of detection of current flow cytometry hardware (90-200 nm). In fact, there exists a large discrepancy in both fluorescence intensity and refractive index (RI) between most calibration beads and biological nanoparticles of interest (REF). As such, a fluorescently tagged virus was chosen as the standard, which allowed us to optimize the instrument settings on a biologically relevant particle. Alternatively, other groups have been successful at analyzing nanoparticles through conjugation with fluorescent beads. The benefits of these bead conjugates are the improved detection capabilities on a wide range of flow cytometers, however it may also introduce a bias into data collection. Most notably, non-bound particles may be below the threshold of the detectors. Additionally, our group has previously demonstrated that antibody labeling of nanoparticles can induce aggregation (Tang et al., 2016). For this reason, the NFC study was carried on viruses having undergone a minimal amount of manipulation, which was limited to being filtered and diluted.

There are advantages and disadvantages in selecting light scatter as the threshold parameter. On the one hand it allows for the detection of all non-fluorescent particles, but on the other, it increases the overall abort rate, at least with our instrument and acquisition software. This increased abort rate resulted in a 3-fold reduction in the total eGFP+ events in comparison to fluorescence thresholding (FIGS. 13 and 14). The choice between SSC and FL for threshold is therefore application dependant. In an experiment where the enumeration of a population of interest is required, samples should be collected using FL-thresholding; the caveat being that this population must be fluorescently labeled. On the other hand, if the goal of the study is to analyze different populations labeled with mutually exclusive fluorescent marker combinations, then SSC thresholding should be chosen to collect all relevant data, and better appreciate small differences in population relative sizes, clustering and distributions.

When analyzing MLVeGFP viruses that contain approximately 100 Env-eGFP molecules on its surface⁶⁰, the greatest gain in fluorescent particle resolution was observed when the power of the 488 nm laser increases from 100 mW to 200mW (FIGS. 16 and 19). Nonetheless, for the detection of antigens at the surface of nanoparticles that are less abundant than Env-eGFP, like for example gp120 on the surface of HIV-1 virions, the higher power laser will likely greatly improve signal resolution.

Retroviruses share many physical properties with EVs such as buoyant density and refractive index due to their overlapping biochemical composition and size. Yet despite these similarities, MLVs as a population are distinct from EVs in that they are slightly more homogeneous in light scattering properties (FIG. 6), and in the way they uptake lipophilic dyes such as DiD (FIG. 33, Panel (C)), and they also appear to contain more total nucleic acids (FIG. 33, Panel (E)). These small differences are important in the context that EVs will contaminate even the purest of retrovirus preparations. Furthermore, given that EVs are known to harbour viral proteins and nucleic acid fragments, and share numerous surface markers, it is most challenging to discriminate them using biochemical approaches. If single-particle characterizations are carried out on viruses, it is essential to develop methods to discriminate them from EVs.

In this work, it is clearly demonstrated that a fluorescent tag on the viral envelope glycoprotein can serve as an excellent identifier. While providing some specificity to detect viral particles, our MLVeGFP virus enable us to evaluate the efficacy of other staining methods. As evidenced by FIGS. 30 and 31, fluorescent labelling of the virus with dyes had a severe impact on its infectivity. Even DHPE-Rhodamine, which stained viral populations very poorly, had a significant impact on viral infectivity. The data presented herein indicates that membrane dyes are imposing a defect at egress in the producer cells or are directly affecting the infected cell metabolism (FIG. 31). It is clear that these methods are not suitable for some downstream applications that require infectious virus, and caution should be taken when analyzing viral fitness in stained particles.

While there was success in staining our viral population, there was a clear overlap with the eGFP⁻ EV population. To further distinguish populations, the relative uniformity of nucleic acid packaging in viruses was taken advantage of. Though MLVeGFP was unable to be resolved from EVs by lipid and nucleic acid staining, it did show superior particle homogeneity (FIG. 33, Panels (C), (F) and (G)). However, VV which has a much larger physical size (360 nm) than MLVs and most EVs in our samples, is clearly resolved from EVs with a combination of nucleic acid dye by fluorescence and SSC analysis (FIG. 33, Panel (I)). In fact, SYBR I staining of VV reveals the presence of viral genomes in both lipid+ (population 4) and lipid− (population 3) populations. This may be indicative of the multiple types of VV subspecies produced by an infected cell, which include VV particles with either a single layer or bilayer lipid envelope. Overall, our assays have shown that it is possible to distinguish enveloped viruses from EVs.

With technical advancements in flow cytometry it is possible to now visualize nanoparticles down to 120 nm or 90 nm using NFC. This innovation now opens a wide array of new possibilities that result from single particle analysis of viruses, but also of EVs. These applications can include the individual profiling of surface antigens, sorting of particles with distinct markers, or even the precise enumeration of particles displaying certain fluorescence of light scattering characteristics. NFC has the potential to bring new understanding to the fields of virology and EV research, as it provides a tool to answer questions that were not previously possible to address.

As described herein, mutant murine leukemia virus with a G->A mutation at position 836 (a numbering convention for the provirus, corresponding to position 95 of GenBank Accession NC_001501) and an eGFP coding sequence inserted into the proline-rich region of the env gene22 produces the fluorescent MLVeGFP(CTG). These fluorescent viruses can be used as biological size standards to calibrate and set-up flow cytometers for the analysis of fluorescently labeled particles, including biological particles such as EVs and viruses, in the 90-200 nm size range.

### EXAMPLE 2

### Overview

Flow cytometry, or more specifically nanoscale flow cytometry (NFC) is becoming a method of choice for the phenotypic analysis of extracellular vesicles (EVs). EVs can range in size from approximately 50 nm to 1 μm in diameter, which places them at the lower end limit of detection for the majority of commercial flow cytometers. Optimization of flow cytometer settings for the analysis of EVs can therefore be challenging. Reference materials such as fluorescently labeled polystyrene or silica beads are often used in the optimization of flow cytometer settings at the acquisition stage. However, synthetic beads have a higher refractive index and often display much more intense fluorescence than biological samples of equivalent size, thereby skewing baseline cytometer settings outside the range of fluorescent EV samples. Here we demonstrate the utility of an eGFP-tagged enveloped virus as a reference material for instrument set-up. Optimization of detector gain and threshold for side-scatter (SSC) and fluorescence was compared using an eGFP-tagged mouse leukemia virus (124 nm in size as established by EM), and the Apogee Bead Mix—a mix of silica (not fluorescent) and polystyrene (fluorescent) beads (110 nm-585 nm). EVs isolated from urine (80 nm median size measured by NTA) and cell-culture media (HUVEC, 67 nm median size measured by NTA) labeled with DiO were analyzed by NFC. EV counts and MFI obtained through instrument settings based on beads vs. virus were compared. Overall use of eGFP-tagged virus for instrument set-up enabled the detection of 10-fold more UVEC EVs and 8-fold more urine-derived EVs.

### Materials and Methods

### Flow Cytometry

Flow cytometry is carried out on a Beckman Coulter Cytoflex S.

### Virus Inactivation and Stabilization: Optimized and Final Conditions

Fixation: Viruses in the cell supernatant were fixed by adding 2% formaldehyde for 1 h at RT.

Dialysis to remove formaldehyde: 3 ml of fixed virus were then dialysed in 3 L of 0.1 μm-filtered PBS for 17 h at 4° C. using a 3.5 kD dialysis membrane (Spectrum Laboratories, CA, USA).

Stabilization: Virus was then mixed at a ratio of 1:1 with 10% (w/v) 0.1 μm-filtered sorbitol. 200 μl aliquots were frozen in 1 ml glass vials sealed with perforated parafilm at −80° C.

Lyophilization: Frozen samples were then lyophilized on dry-ice for 18 h at a pressure range from 0.019 to 0.031 mbar. For reconstitution, 200 μl of 0.1 μm-filtered MiliQ H2O was added to lyophilized sample.

### Nanoparticle Tracking Analysis (NTA)

NTA is carried out using a Zeta View PMX110. Setup with 105 and 500 nm polystyrene beads, analysis with 11 camera positions, 2 s video length, 30 fps @21° C.

### Extracellular Vesicles (EVs)

EVs are obtained with the following protocol:

Spin 50 ml of human urine/HUVEC cell culture supernatants @2500 g for 10min to remove cellular debris

Spin supernatant 20,000 g for 20 min @4° C. to obtain microparticle pellet (MP), keep supernatant (SN1)

Resuspend MP in isolation solution (250 mM sucrose, 10 mM triethanolamine, pH7.6) with 200 mg/ml of DTT, incubate at room temperature for 10 min. Spin @20,000 g for 20 minutes, keep supernatant (SN2).

Pool SN1 and SN2 (urine only), spin at 100,000 g for 90 min to obtain exosome pellet.

Resuspend exosomes in 0.1 μm-filtered PBS

### Labelling

For labelling, Dio solution is directly added to 300 μl of resuspended exosomes (10 μg) to obtain a final concentration of 10 μM, and incubated for 1.5 hour at 37° C. Exosomes are spun down at 100,000 g for 90 minutes (4° C.), resuspended in PBS

### Viruses

Mouse leukemia virus (MLV-GFP): Virus collected from supernatants of chronically infected NIH 3T3 cells in serum & phenol red-free DMEM, diluted in 0.1 μm-filtered PBS

Avian leukosis virus (ALV-sfGFP): 293T cells were transfected with RCASb for 36 hrs, cells were washed and re-plated in serum & phenol red-free DMEM, virus was produced for 3 hrs, supernatants were collected and then diluted in 0.1 μm-filtered PBS

### Results and Discussion

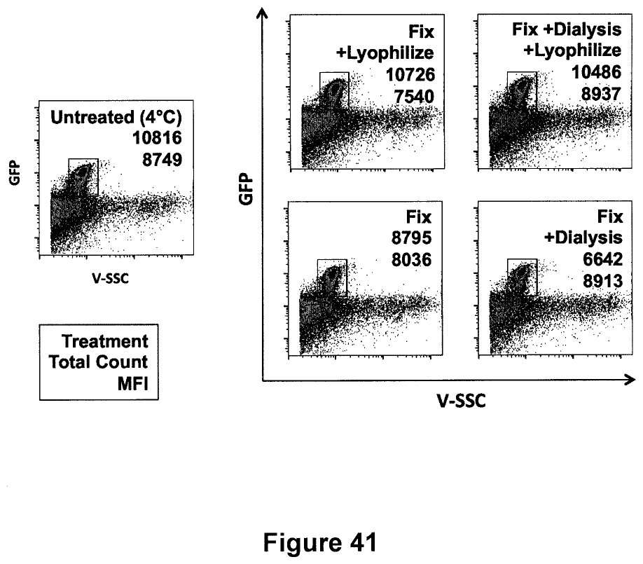

FIG. 41 depicts the results of experiments involving sample fixation and dialysis. Sample preservation methods to stabilize particle counts and median fluorescence intensity of eGFP in the virus population. Fixation with formalin abrogates infectivity of MLVeGFP. The best retention of both particle count as well as eGFP fluorescence was achieved when fixed viruses were dialyzed followed by lyophilisation. This process removed excess formalin and preserved the virus particles efficiently.

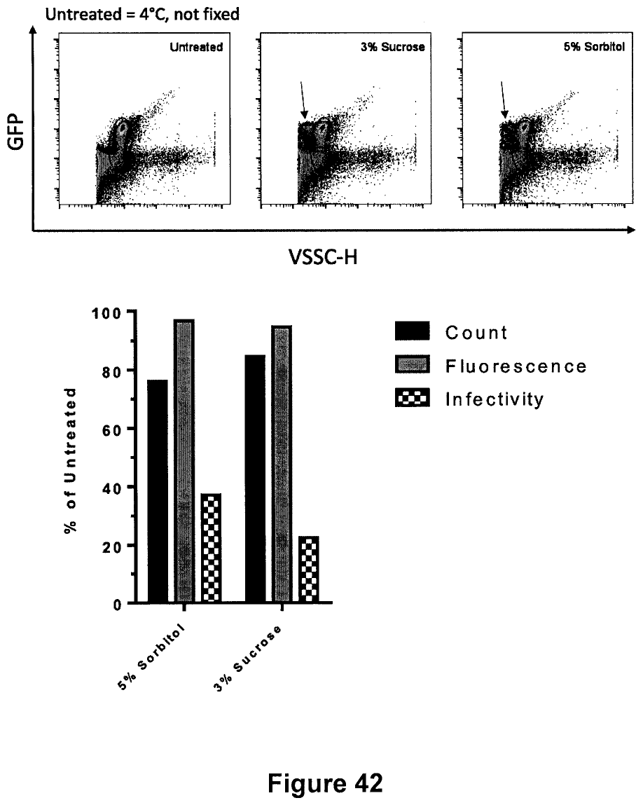

FIG. 42 examines the use of stabilizers to maintain particle count and eGFP fluorescence of the virus. Result indicate that sorbitol 5% and sucrose 3% similarly preserved virus count and eGFP fluorescence (based on events in the black gate), but sorbitol appeared to have less of a negative impact on virus infectivity and resulted in fewer damaged particles (population beside black gate population denoted by an arrow). Virus infectivity is taken as an indirect indication of the intactness of viral surface proteins since these receptors are required for binding, entry, and infection of a cell. Sorbitol 5% is therefore considered the slightly better option.

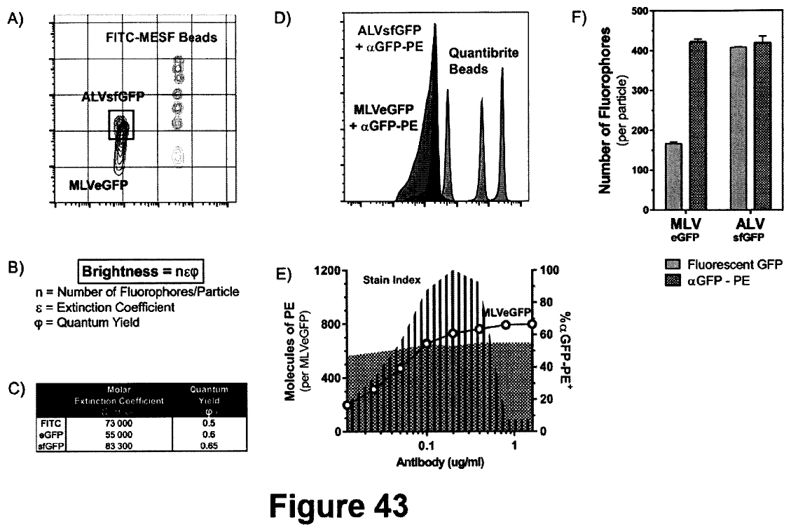

FIG. 43 shows enumeration of antigen expression on the virus surface by two methods. The ability to profile and quantify viral antigens expressed on the surface of viruses and EVs is the ultimate goal of nanoscale flow cytometry. Here we were able to correlate measured fluorescence with the number of eGFP molecules using two approaches: 1) Direct: fluorescence from eGFP expressed on the surface of viruses, and 2) indirect:

fluorescence from Anti-GFP antibodies conjugated with various fluorophores. Since the viruses are homogeneous in size, they are readily detected using light scatter (side scatter or SSC) and green fluorescence.

Method 1: NIST (National Institute of Standards and Technology) certified FITC-MESF (FITC molecules of equivalent soluble fluorophores) beads were run with MLVeGFP and ALVsfGFP and used as a reference for green fluorescence (A). Each bead population is associated with a specific number of FITC molecules. By correlating the median fluorescence intensity of green fluorescence of each bead population with the number of associated FITC molecules, the equivalent FITC molecules of fluorescence of both MLVeGFP and ALVsfGFP was extrapolated by linear regression. The fluorescence associated with the equivalent FITC molecules of both virus populations was converted to molecules of eGFP and superfolder GFP (sfGFP). This was done through a calculation for particle brightness (B) as a function of the extinction coefficient and the quantum yield of each fluorophore. The values for FITC, eGFP, and sfGFP are published (C).

Method 2: Quantibrite PE beads (BD Biosciences) were run with MLVeGFP and ALVsfGFP viruses labeled with anti-GFP-PE antibody (D). Quantibrite PE beads are similar to the FITC MESF beads in that each of the three populations are associated with a specific number of PE molecules. Antibody concentrations for virus labeling were determined by titration and the stain index was determined. Molecules of PE were extrapolation by linear regression using the median fluorescence intensities in correlation to PE molecules in each bead population. Because there is a range of antibody concentrations where all positive viruses are labeled (i.e. epitope saturation), the average between the highest and lowest PE molecules per virus was taken.

Using Method 1, we find that eGFP expression on MLV is about 150 molecules of fluorescent eGFP per virus particle and ALV expresses on the order of 400 molecules of sfGFP. The number of sfGFP expressed by ALV is validated with Method 2.

Method 2 estimates close to 400 eGFP expressed on MLVeGFP, which appears to be an over estimation. However eGFP is known to oligomerization in secretory pathway of cells to form non-fluorescent mixed disulfides, while sfGFP does not. Since viruses are produced through this pathway it is very likely that not all eGFP expressed on MLVeGFP are fluorescent. These non-fluorescent eGFP will still be detected by the anti-GFP antibody. Since Method 2 is dependent on antibody detection of GFP molecules and not GFP fluorescence itself, it is feasible that this method will detect more GFP molecules in MLVeGFP than Method 1 which relies on the fluorescence of the fluorophore.⁶⁸ The finding of approximately 400 molecules of GFP per virus also correlates with values reported in literature. Expression of the eGFP molecule is associated with the MLV envelope glycoprotein gp85. Three gp85 proteins get cleaved to form a trimeric envelope protein spike on the surface of the virus. CryoEM studies to enumerate envelope glycoprotein spikes on MLV have found the expression level to be on the order of 120 spikes per virus. Since there should be 3 eGFP per envelope glycoprotein spike, our estimate of 400 eGFP molecules means approximately 130 spikes per virion.

Thus, by comparing to a NIST standard having defined number of fluorescent molecules, the number of molecules of the fluorescent marker on the virus control can be deduced by linear regression. The virus controls can thereafter act as NIST surrogates, in that they can be used to enumerate markers on the surface of microparticles (e.g., EVs or viruses). An advantage of using the virus controls described herein over NIST standards is that NIST standards are quite bright and contain much higher numbers of molecules than what is at the surface of the virus controls and other microparticles to be measured. Using a fluorescent standard that is closer in fluorescence to the particles being measured increases accuracy. The virus controls described herein would therefore, in some embodiments, be more suitable than NIST standards to measure low levels of fluorescence on microparticles and enumerate markers, receptors, etc.

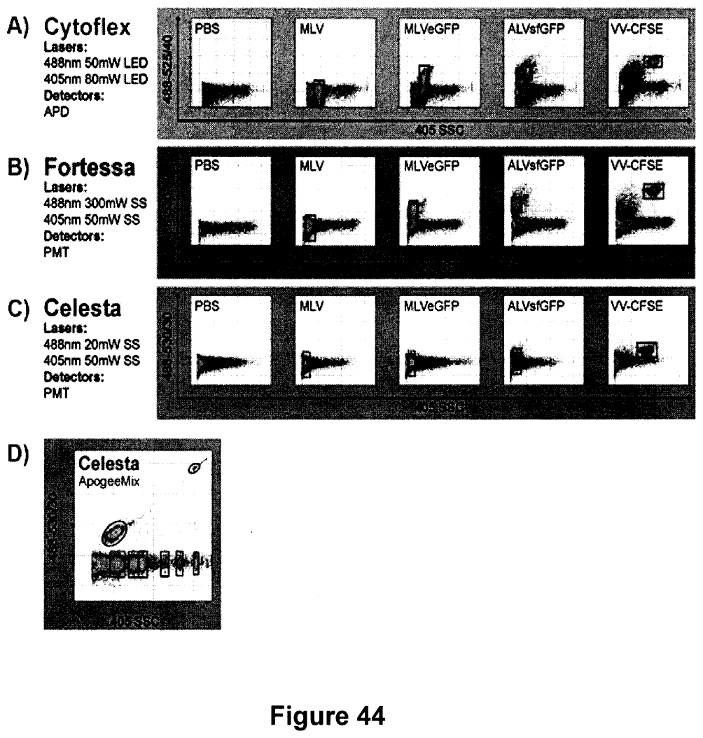

FIG. 44 depicts use of virus particles to test instrument fluorescence sensitivity (Application 1). Retroviruses MLV (non-fluorescent), MLVeGFP, and ALVsfGFP as well as CFSE labeled vaccinia virus were analysed using the Cytoflex S (A), LSRFortessa (Fortessa) (B), and FACSCelesta (Celesta) (C). The Cytoflex and Fortessa are instruments that are known to have small particle detection capabilities. Although they each employ different types of detectors as well as lasers of different power, virus populations can be resolved from both SSC and green (when applicable) on both The Cytoflex and Fortessa. The Celesta is a conventional flow cytometer with lower powered lasers designed for the analysis of cells and is generally not considered as a nanoscale flow cytometry instrument. This is demonstrated by the inability to resolve the GFP fluorescence of MLVeGFP and ALVsfGFP (C, dotted red gates). The ApogeeMix is a commercially available reference bead mix used for nanoscale flow cytometry. The mixture of silica and fluorescence polystyrene beads can be used to gauge side-scatter (SSC) resolution and green fluorescence detection (488-530/30) (Populations: Polystyrene green 1) 110 nm polystyrene green, 2) 500 nm, Silica 3) 180 nm, 4) 240 nm, 5) 300 nm, 6) 590 nm, 7) 880 nm, 8) 1300 nm). The smaller bead populations, 110 nm polystyrene (1) and 180 nm silica (3) are clearly resolved on the Celesta, but as demonstrated in C), resolution of bead standards does not translate to detection of biological particles in a similar size range.

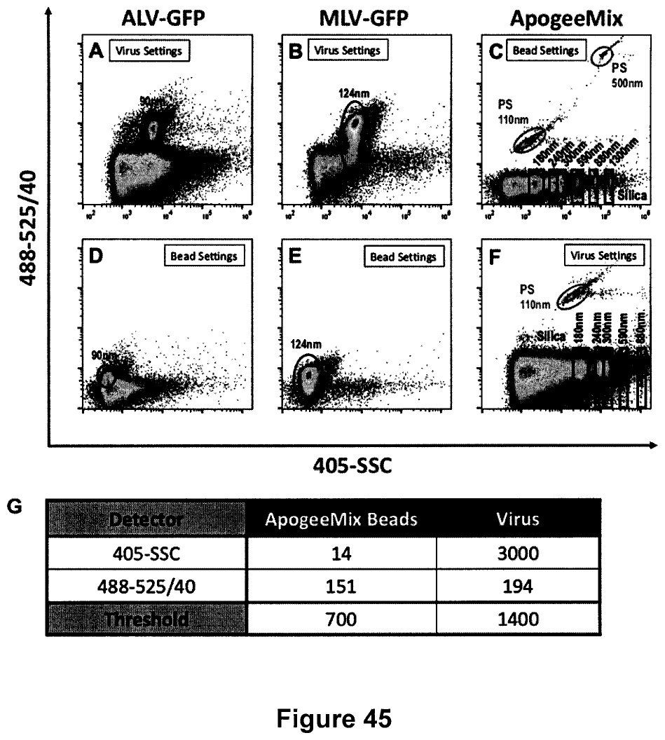

FIG. 45 depicts comparison of instrument settings determined using beads (ApogeeMix) with settings optimized with MLVeGFP and ALVsfGFP for analysis of extracellular vesicles by nanoscale flow cytometry (Application 1, continued).

A&B) Detector gain and threshold were optimized for analysis of MLVeGFP and ALVsfGFP viruses which are 124 nm and 90 nm by EM, respectively. C) Settings were similarly optimized to display all ApogeeMix bead populations (polystyrene-PS, and silica). D&E) ALVsfGFP and MLV-GFP samples were re-run using bead optimized settings. F) ApogeeMix beads were re-run with virus optimized settings. Events were collected for 1 min at 10 ul/min. G) Detector gain and threshold values for virus and beads (ApogeeMix) settings. Viruses are largely undetectable when the instruments are set-up using ApogeeMix beads.

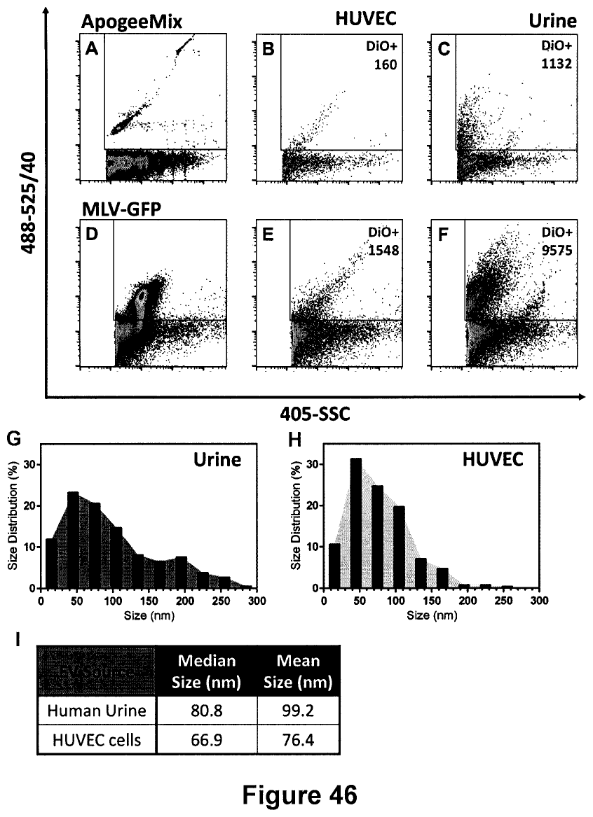

FIG. 46 depicts particle counts of DiO-labeled extracellular vesicles analyzed using bead and virus optimized setting (Application 1 continued). A) ApogeeMix beads, B) HUVEC cell supernatant-derived EVs, and C) Urine-derived EVs analyzed using bead optimized detector gains and threshold. D) MLV-eGFP, E) HUVEC cell supernatant derived EVs, and F) Urine-derived EVs analyzed using MLV-eGFP optimized detector gains and threshold. Total particle counts for DiO+ EVs obtained using bead vs. virus optimized settings and gates are displayed on the top-right of each panel. Events were collected for 1 min at 10 μl/min. Size distribution of extracellular vesicles were measured. G) Human urine, and H) HUVEC cell culture supernatant-derived EVs were analysed by nanoparticle tracking. I) Mean and median diameter size of urine and HUVEC EVs. The use of eGFP-tagged virus for instrument set-up enabled the detection of 10-fold more UVEC EVs and 8-fold more urine-derived EVs.

FIG. 47 depicts use of virus particles as positive controls for extracellular vesicle labeling and surface antigen profiling (Application 2).

MLV (non-fluorescent), MLVeGFP, and ALVsfGFP and vaccinia virus were labeled with a combination of anti-CD81, -CD63, and -CD9 antibodies. CD81, CD63, and CD9 are all part of a family of transmembrane proteins known as tetraspanins. Tetraspanins play a role in the cellular endocytic pathway, which is implicated in the production of exosomes and are currently used as identifying markers for extracellular vesicles, specifically exosomes. Retroviruses share many biosynthetic pathways with extracellular vesicles for particle production. Tetraspanins have been found to be associated with the retrovirus protein (Gag) in virus assembly and release.

Violet Proliferation Dye 450 is an esterase sensitive dye used for labeling cellular amine groups. This dye is only fluorescent when cleaved by esterases and therefore is dependent on the presence of intracellular esterases in the particle to be labeled. It is also is currently one of the dyes used to identify microparticles, a subpopulation of extracellular vesicles. Since microparticles are believed to be produced by budding from the surface of cells, and are in general larger than exosomes, it is thought that more cellular esterases are packaged into these extracellular vesicles, which then allows for them to be identified by esterase-sensitive dyes. The production of microparticles is also associated with cell death such as apoptosis and necrosis. Vaccinia virus buds through the cell surface membrane and induces cell lysis through apoptosis and necrosis.

FIG. 46, panels A to C show tetraspanin and Annexin V expression on MLVeGFP and ALVsfGFP. Tetraspanins (CD63, CD81, and CD9) are expressed on both MLVeGFP and ALVsfGFP. MLV is MLVeGFP without eGFP and is used as a negative staining control for the anti-GFP antibody. Buffer samples with antibody as used as a control for background events. None of the retroviruses express detectable levels of Annexin V.

FIG. 46, panel D examines the presence of cellular esterases in Vaccinia virus. Vaccinia virus is identified by SSC and FITC labeled anti-Vaccinia antibody. The virus population gated from D) is show to be positive for Violet Proliferation Dye 450, which indicates a presence of cellular esterases in vaccinia virus.

These findings suggest that retroviruses (MLV and ALV) share identifying markers for extracellular vesicles, making them an ideal positive control particle for EV labelling assays for flow cytometry, some of which will be discussed.

Markers on the surface of viruses and EVs are indicative of the cell type that is releasing these particles and also, these markers provide a glimpse at the state/heath of a cell (e.g., healthy, dying, necrotic, stressed, cancerous, etc.).

For example, markers on the surface of HIV virions released from infected patients can be profiled in the context of latency reversal therapy that has the objective of purging HIV reservoirs. Reservoirs are cells infected with HIV that don't necessarily release virus, but that may be stimulated to do so (for example when a patient has another simultaneous infection). The reservoirs are elusive, and not all ell types that harbour latent virus have yet been identified. The first viruses released during latency reversal will bear the hallmarks (surface markers) of their parental cell. These markers and receptors on the viruses can be profiled, permitting the reservoir cells to be identified.

For influenza virus, some virus subtypes infect the upper or lower respiratory tract. These often correlate with severity of disease. It is possible to harvest blood plasma from an infected person and profile markers on influenza to quickly determine the site of the infection and the health of the infected epithelial cells.

As a further example, dying virus-infected cells express apoptosis markers (ie. Annexin V) on their surface. If the virus also expresses these markers, the severity of the infection can be deduced.

For diagnostic applications pertaining to extracellular vesicles, plasma could be screened EVs based on the expression of surface markers related to a specific disease. This could help early diagnosis and prognostic of liver and kidney disease for example, also cancer, diabetes.

In all these examples, a positive and negative control of similar size as the particles of interest (EVs or viruses) must be used to calibrate the flow cytometer. Fluorescent virus standards, described herein in some embodiments, can be designed to express any given marker. This may be achieved by serendipitous hijacking of a marker of interest when the virus is released from a specific cell type. The marker may be endogenous or exogenous to the cell type. This may also be achieved by engineering surface markers to be specifically captured by egress virus. This can be done by inserting (e.g., by cloning) the transmembrane (TM) domain of the native viral envelope glycoprotein to the membrane-bound extremity of the surface marker of interest. When the recombinant protein is expressed at the surface of the cell (by transfection or retroviral expression), the egress virus will uptake the marker of interest as it will think that it is its own glycoprotein. Fluorescent virus standards expression such proteins would be useful positive controls in diagnostic-type applications.

### REFERENCES

28 Arakelyan, A., Fitzgerald, W., Margolis, L. & Grivel, J. C. Nanoparticle-based flow virometry for the analysis of individual virions. J Clin Invest 123, 3716-3727, doi:10.1172/JCI67042 (2013).

29 Bonar, M. M. & Tilton, J. C. High sensitivity detection and sorting of infectious human immunodeficiency virus (HIV-1) particles by flow virometry. Virology 505, 80-90, doi:10.1016/j.viro1.2017.02.016 (2017).

30 Brussaard, C. P. D., Marie, D. & Bratbak, G. Flow cytometric detection of viruses. J Virol Methods 85, 175-182, doi:Doi 10.1016/S0166-0934(99)00167-6 (2000).

31 Gaudin, R. & Barteneva, N. S. Sorting of small infectious virus particles by flow virometry reveals distinct infectivity profiles. Nature communications 6, 6022, doi:10.1038/ncomms7022 (2015).

32 Hercher, M., Mueller, W. & Shapiro, H. M. Detection and discrimination of individual viruses by flow cytometry. J Histochem Cytochem 27, 350-352, doi:10.1177/27.1.374599 (1979).

33 Marie, D., Brussaard, C. P. D., Thyrhaug, R., Bratbak, G. & Vaulot, D. Enumeration of marine viruses in culture and natural samples by flow cytometry. Appl Environ Microbiol 65, 45-52 (1999).

34 Musich, T. et al. Flow virometric sorting and analysis of HIV quasispecies from plasma. JCI Insight 2, e90626, doi:10.1172/jci.insight.90626 (2017).

35 Steen, H. B. Flow cytometer for measurement of the light scattering of viral and other submicroscopic particles. Cytometry A 57, 94-99, doi:10.1002/cyto.a.10115 (2004).

36 Tang, V. A. et al. Single-particle characterization of oncolytic vaccinia virus by flow virometry. Vaccine 34, 5082-5089, doi:10.1016/j.vaccine.2016.08.074 (2016).

37 Loret, S., El Bilali, N. & Lippe, R. Analysis of herpes simplex virus type I nuclear particles by flow cytometry. Cytometry A 81, 950-959, doi:10.1002/cyto.a.22107 (2012).

38 van der Pol, E., Boing, A. N., Harrison, P., Sturk, A. & Nieuwland, R. Classification, functions, and clinical relevance of extracellular vesicles. Pharmacological reviews 64, 676-705, doi:10.1124/pr.112.005983 (2012).

39 Robbins, P. D. & Morelli, A. E. Regulation of immune responses by extracellular vesicles. Nature reviews. Immunology 14, 195-208, doi:10.1038/nri3622 (2014).

40 Arakelyan, A. et al. Flow virometry analysis of envelope glycoprotein conformations on individual HIV virions. Sci Rep 7, 948, doi:10.1038/s41598-017-00935-w (2017).

41 Arakelyan, A. et al. Flow Virometry to Analyze Antigenic Spectra of Virions and Extracellular Vesicles. J Vis Exp, doi:10.3791/55020 (2017).

42 Arraud, N., Gounou, C., Linares, R. & Brisson, A. R. A simple flow cytometry method improves the detection of phosphatidylserine-exposing extracellular vesicles. J Thromb Haemost 13, 237-247, doi:10.1111/jth.12767 (2015).

43 Arraud, N., Gounou, C., Turpin, D. & Brisson, A. R. Fluorescence triggering: A general strategy for enumerating and phenotyping extracellular vesicles by flow cytometry. Cytometry A 89, 184-195, doi:10.1002/cyto.a.22669 (2016).

44 Inglis, H., Norris, P. & Danesh, A. Techniques for the analysis of extracellular vesicles using flow cytometry. J Vis Exp, doi:10.3791/52484 (2015).

45 Inglis, H. C. et al. Techniques to improve detection and analysis of extracellular vesicles using flow cytometry. Cytometry A 87, 1052-1063, doi:10.1002/cyto.a.22649 (2015).

46 Morales-Kastresana, A. et al. Labeling Extracellular Vesicles for Nanoscale Flow Cytometry. Sci Rep 7, 1878, doi:10.1038/s41598-017-01731-2 (2017).

47 Stoner, S. A. et al. High sensitivity flow cytometry of membrane vesicles. Cytometry A 89, 196-206, doi:10.1002/cyto.a.22787 (2016).

48 Vagida, M. et al. Flow analysis of individual blood extracellular vesicles in acute coronary syndrome. Platelets 28, 165-173, doi:10.1080/09537104.2016.1212002 (2017).

49 Zicari, S. et al. Evaluation of the maturation of individual Dengue virions with flow virometry. Virology 488, 20-27, doi:10.1016/j.viro1.2015.10.021 (2016).

50 Erlwein, O., Buchholz, C. J. & Schnierle, B. S. The proline-rich region of the ecotropic Moloney murine leukaemia virus envelope protein tolerates the insertion of the green fluorescent protein and allows the generation of replication-competent virus. J Gen Virol 84, 369-373, doi:10.1099/vir.0.18761-0 (2003).

51 Langlois, M. A., Kemmerich, K., Rada, C. & Neuberger, M. S. The AKV murine leukemia virus is restricted and hypermutated by mouse APOBEC3. J Virol 83, 11550-11559, doi:10.1128/JVI.01430-09 (2009).

52 Sliva, K., Erlwein, O., Bittner, A. & Schnierle, B. S. Murine leukemia virus (MLV) replication monitored with fluorescent proteins. Virol J 1, 14, doi:10.1186/1743-422X-1-14 (2004).

53 Carroll, M. W. & Moss, B. Host range and cytopathogenicity of the highly attenuated MVA strain of vaccinia virus: propagation and generation of recombinant viruses in a nonhuman mammalian cell line. Virology 238, 198-211, doi:10.1006/viro.1997.8845 (1997).

54 Heo, J. et al. Randomized dose-finding clinical trial of oncolytic immunotherapeutic vaccinia JX-594 in liver cancer. Nat Med 19, 329-336, doi:10.1038/ nm.3089 (2013).

55 Parato, K. A. et al. The oncolytic poxvirus JX-594 selectively replicates in and destroys cancer cells driven by genetic pathways commonly activated in cancers. Mol Ther 20, 749-758, doi:10.1038/mt.2011.276 (2012).

56 Zeh, H. J. et al. First-in-man study of western reserve strain oncolytic vaccinia virus: safety, systemic spread, and antitumor activity. Mol Ther 23, 202-214, doi:10.1038/mt.2014.194 (2015).

57 Roy, D. G. et al. Programmable insect cell carriers for systemic delivery of integrated cancer biotherapy. J Control Release 220, 210-221, doi:10.1016/j.jconre1.2015.10.030 (2015).

61 Linares, R., Tan, S., Gounou, C., Arraud, N. & Brisson, A. R. High-speed centrifugation induces aggregation of extracellular vesicles. J Extracell Vesicles 4, 29509, doi:10.3402/jev.v4.29509 (2015).

62 Gray, W. D., Mitchell, A. J. & Searles, C. D. An accurate, precise method for general labeling of extracellular vesicles. MethodsX 2, 360-367, doi:10.1016/j.mex.2015.08.002 (2015).

63 San-Juan-Vergara, H. et al. Cholesterol-rich microdomains as docking platforms for respiratory syncytial virus in normal human bronchial epithelial cells. J Virol 86, 1832-1843, doi:10.1128/JVI.06274-11 (2012).

64 Zhang, S., Chan, K. R., Tan, H. C. & Ooi, E. E. Dengue virus growth, purification, and fluorescent labeling. Methods Mol Biol 1138, 3-14, doi:10.1007/978-1-4939-0348-1_1 (2014).

65 Xu, H. et al. Real-time Imaging of Rabies Virus Entry into Living Vero cells. Sci Rep 5, 11753, doi:10.1038/srep11753 (2015).

66 Lakadamyali, M., Rust, M. J., Babcock, H. P. & Zhuang, X. Visualizing infection of individual influenza viruses. Proc Natl Acad Sci USA 100, 9280-9285, doi:10.1073/pnas.0832269100 (2003).

67 Yaeger, M. et al. Supramolecular organization of immature and mature murine leukemia virus revealed by electron cryo-microscopy: Implications for retroviral assembly mechanisms. Proc Natl Acad Sci USA 95, 7299-7304.

68 Jain, R. et al. Oligomerization of green fluorescent protein in the secretory pathway of endocrine cells. Biochem. J. 360, 645-649 (2001).

All references cited herein are expressly incorporated by reference.

In the preceding description, for purposes of explanation, numerous details are set forth in order to provide a thorough understanding of the embodiments. However, it will be apparent to one skilled in the art that these specific details are not required. In other instances, well-known electrical structures and circuits are shown in block diagram form in order not to obscure the understanding. For example, specific details are not provided as to whether the embodiments described herein are implemented as a software routine, hardware circuit, firmware, or a combination thereof.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. A recombinant nucleic acid encoding a modified gammaretrovirus the recombinant nucleic acid comprising:
a mutation that reduces or abolishes expression of the viral glyco-Gag protein, and
a nucleic acid encoding a fluorescent protein inserted in-frame into the proline-rich region (PRR) of the viral env protein.

2. The recombinant nucleic acid of claim 1, wherein the modified gammaretrovirus is CAS-BR-E, MLV 1313 (Amphotropic MLV), Pmv11 (Polytropic MLV), Xmx15 (Xenotropic MLV), FrMLV (Friend MLV), M-MLV (Moloney MLV), DG-75, AKV MLV (AKV MLV), SL3-3 MLV, E-MLV (Ecotropic MLV), Rauscher MLV, Mus Dunni endogenous virus, Abelson MLV, XMRV, Porcine endogenous type C, Gibbon leukemia virus, Baboon endogenous virus strain M7, Feline leukemia virus, Koala retrovirus, or Wooly monkey virus.

3. The recombinant nucleic acid of claim 1 or 2, wherein the modified gammaretrovirus is a modified Moloney murine leukemia virus (M-MLV), wherein the mutation reduces or abolishes expression of gPr80 protein.

4. The recombinant nucleic acid of claim 3, wherein the recombinant nucleic acid comprises a nucleic acid sequence derived from GenBank Accession NCBI: NC_001501, modified with said mutation and said nucleic acid encoding fluorescent protein.

5. The recombinant nucleic acid of claim 3, wherein the recombinant nucleic acid comprises the nucleic acid sequence of GenBank Accession NCBI: NC_001501, modified with said mutation and said nucleic acid encoding fluorescent protein.

6. The recombinant nucleic acid of claim 5, wherein the mutation that reduces or abolishes expression of gPr80 is in the CTG codon at positions 93-95.

7. The recombinant nucleic acid of claim 6, wherein the mutation that reduces abolishes expression of gPr80 is a CTG→CTA mutation.

8. The recombinant nucleic acid of any one of claims 1 to 7, wherein the PRR of the viral env protein corresponds to the region encoded by nucleotide positions 6302 to 6433 of GenBank Accession NCBI: NC_001501.

9. The recombinant nucleic acid of any one of claims 1 to 8, wherein the fluorescent protein is inserted into the PRR at a position corresponding to the position after the serine at position 6400.

10. A vector comprising the recombinant nucleic acid of any one of claims 1 to 9.

11. A host cell comprising the recombinant nucleic acid of any one of claims 1 to 9, or the vector according to claim 10.

12. The host cell according to claim 11, wherein the host cell comprises at least one selected marker.

13. The host cell according to claim 12, wherein the at least one selected marker comprises a plurality of markers of a selected profile.

14. An enveloped virus particle produced by the host cell according to claim 11.

15. An enveloped virus particle produced by the host cell according to claim 12, wherein the enveloped virus comprises the at least one selected marker.

16. An enveloped virus particle produced by the host cell according to claim 13, wherein the enveloped virus comprises the plurality of markers of the selected profile.

17. A use of enveloped virus particles, wherein the virus particles are fluorescent, as a size or calibration standard in nanoscale flow cytometry.

18. The use of claim 17, wherein the nanoscale flow cytometry is for measurement of particles less than 200 nm in size.

19. The use of claim 17 or 18, wherein the enveloped virus particles each comprise fluorescent dye.

20. The use of claim 17 or 18, wherein the enveloped virus particles each comprise one or more fluorescent protein.

21. The use of claim 20, wherein the fluorescent protein is enhanced green fluorescent protein (eGFP).

22. The use of claim 20 or 21, wherein the enveloped virus particles each comprise viral envelope proteins, each labelled with the fluorescent protein.

23. The use of claim 22, wherein the viral envelope proteins are M-MLV envelope proteins.

24. The use of claim 22 or 23, wherein the enveloped virus particles are pseudotyped with the viral envelope proteins.

25. The use of claim 24, wherein the viral envelope proteins labelled with fluorescent protein are as encoded by SEQ ID NO: 1.

26. The use of claim 24 or 25, wherein the pseudotyping is accomplished with a vector comprising SEQ ID NO: 2.

27. The use of any one of claims 24 to 26, wherein the enveloped virus particles are pseudotyped to be non-infectious to humans.

28. The use of any one of claims 24 to 27, wherein the enveloped virus particles are encoded by a nucleic acid comprising at least one sequence modification that reduces or abrogates expression of the endogenous viral envelope proteins.

29. The use of any one of claims 17 to 28, wherein the enveloped virus particles comprise MLV, ALV, HIV, HSV, Influenza, and/or VSV particles.

30. The use of any one of claims 17 to 28, wherein the enveloped virus particles comprise gammaretrovirus particles.

31. The use of claim 30, wherein the gammaretrovirus is CAS-BR-E, MLV 1313 (Amphotropic MLV), Pmv11 (Polytropic MLV), Xmx15 (Xenotropic MLV), FrMLV (Friend MLV), M-MLV (Moloney MLV), DG-75, AKV MLV (AKV MLV), SL3-3 MLV, E-MLV (Ecotropic MLV), Rauscher MLV, Mus Dunni endogenous virus, Abelson MLV, XMRV, Porcine endogenous type C, Gibbon leukemia virus, Baboon endogenous virus strain M7, Feline leukemia virus, Koala retrovirus, or Wooly monkey virus.

32. The use of claims 29 to 31, wherein the enveloped virus particles comprise M-MLV particles.

33. The use of any one of claims 17 to 32, wherein the enveloped virus particles are encoded by the recombinant nucleic acid as defined in any one of claims 1 to 9.

34. The use of any one of claims 17 to 32, wherein the enveloped virus particles comprise a plurality of enveloped virus types, each of the types being a different size.

35. The use of any one of claims 17 to 33, wherein the enveloped virus particles are of a single enveloped virus type.

36. The use of any one of claims 14 to 35, wherein the enveloped virus particles further comprise at least one selected marker.

37. The use of claim 36, wherein the at least one selected marker comprises a plurality of markers of a selected profile.

38. The use of claim 36 or 37, wherein the size or calibration standard is a positive control for detection of viral particles or extracellular vesicles comprising the same at least one selected marker.

39. The use of claim 38, where the at least one selected marker is characteristic of a biological parameter.

40. The use of claim 36 or 37, wherein the size or calibration standard is a control for enumeration of markers on microparticles.

41. A method of calibrating a flow cytometer comprising:
measuring a calibration standard comprising enveloped virus particles, wherein the virus particles are fluorescent, in nanoscale flow cytometry.

42. A flow cytometry method comprising:
measuring a size standard comprising enveloped virus particles, wherein the virus particles are fluorescent, in nanoscale flow cytometry.

43. The method of claim 41 or 42, wherein the enveloped virus particles are as defined in any one of claims 17 to 40.

44. The method of claim 41 or 42, wherein the enveloped virus particles comprise enveloped virus particles encoded by the recombinant nucleic acid according to any one of claims 1 to 9.

45. A size standard or calibration ladder for nanoscale flow cytometry, comprising a plurality of types of enveloped virus particles, each of the enveloped particles being fluorescent, wherein each of the types of virus particles is of a different size.

46. The size standard or calibration ladder of claim 45, wherein the virus particles are as defined in any one of claims 17 to 40.

47. The size standard or calibration ladder of claim 46, wherein the plurality of types of enveloped virus particles comprise enveloped virus particles encoded by the recombinant nucleic acid according to any one of claims 1 to 9.

48. A method of producing fluorescent enveloped virus particles comprising at least one selected marker, the method comprising:
infecting a host cell expressing the at least one selected marker with enveloped virus particles, and
recovering enveloped virus particles produced by the infected host cell, the enveloped virus particles comprising the selected biomarker profile, wherein the recovered enveloped virus particles are fluorescent.

49. The method of claim 48, further comprising labelling the recovered enveloped virus particles with a fluorescent dye.

50. The method of claim 48, wherein the recovered enveloped virus particles each comprise one or more fluorescent protein.

51. The method of claim 50, wherein the fluorescent protein is enhanced green fluorescent protein (eGFP).

52. The method of claim 50 or 51, wherein the enveloped virus particles each comprise viral envelope proteins, each labelled with the fluorescent protein.

53. The method of claim 52, wherein the viral envelope proteins are M-MLV envelope proteins.

54. The method of claim 52 or 53, wherein the enveloped virus particles are pseudotyped with the viral envelope proteins.

55. The method of claim 54, wherein the viral envelope proteins labelled with fluorescent protein are as encoded by SEQ ID NO: 1.

56. The method of claim 54 or 55, wherein the pseudotyping is accomplished with a vector comprising SEQ ID NO: 2.

57. The method of any one of claims 54 to 56, wherein the enveloped virus particles are pseudotyped to be non-infectious to humans.

58. The method of any one of claims 48 to 57, wherein the enveloped virus particles are encoded by a nucleic acid comprising at least one sequence modification that reduces or abrogates expression of the endogenous viral envelope proteins.

59. The method of any one of claims 48 to 58, wherein the enveloped virus particles comprise MLV, ALV, HIV, HSV, Influenza, and/or VSV particles.

60. The method of any one of claims 48 to 59, wherein the enveloped virus particles comprise gammaretrovirus particles.

61. The method of claim 60, wherein the gammaretrovirus is CAS-BR-E, MLV 1313 (Amphotropic MLV), Pmv11 (Polytropic MLV), Xmx15 (Xenotropic MLV), FrMLV (Friend MLV), M-MLV (Moloney MLV), DG-75, AKV MLV (AKV MLV), SL3-3 MLV, E-MLV (Ecotropic MLV), Rauscher MLV, Mus Dunni endogenous virus, Abelson MLV, XMRV, Porcine endogenous type C, Gibbon leukemia virus, Baboon endogenous virus strain M7, Feline leukemia virus, Koala retrovirus, or Wooly monkey virus.

62. The method of claims 59 to 61, wherein the enveloped virus particles comprise M-MLV particles.

63. The method of any one of claims 48 to 62, wherein the enveloped virus particles are encoded by the recombinant nucleic acid according to any one of claims 1 to 9 or the vector according to claim 10.

64. The method of any one of claims 48 to 63, wherein the at least one selected marker is endogenous to the host cell.

65. The method of any one of claims 48 to 64, wherein the at least one selected marker is exogenous to the host cell.

66. The method of any one of claims 48 to 65, wherein the at least one is/are marker characteristic of a disease cell.

67. The method of any one of claims 48 to 65, wherein the at least one selected marker comprises a plurality of markers of a selected profile.

68. The method of claim 67, wherein the profile is characteristic of a disease cell.

69. The method of any one of claims 48 to 68, wherein the at least one selected marker is a recombinant protein that is modified to promote incorporation of the marker into the recovered enveloped virus particles.

70. The method of claim 69, wherein the recombination protein comprises a membrane signal peptide or a transmembrane (TM) domain of a native viral envelope glycoprotein.


SEQUENCE LISTING (2 sequences)

SEQ ID NO: 1 - DNA, 2703 bp, Artificial Sequence
  Pseudotyping Construct CDS
atggcgcgttcaacgctctcaaaaccccttaaaaataaggttaacccgcgaggccccctaatccccttaattcttctgatgctcagaggggtcagtactgcttcgcccggctccagtcctcatcaagtctataatatcacctgggaggtaaccaatggagatcgggagacggtatgggcaacttctggcaaccaccctctgtggacctggtggcctgaccttaccccagatttatgtatgttagcccaccatggaccatcttattgggggctagaatatcaatcccctttttcttctcccccggggcccccttgttgctcagggggcagcagcccaggctgttccagagactgcgaagaacctttaacctccctcacccctcggtgcaacactgcctggaacagactcaagctagaccagacaactcataaatcaaatgagggattttatgtttgccccgggccccaccgcccccgagaatccaagtcatgtgggggtccagactccttctactgtgcctattggggctgtgagacaaccggtagagcttactggaagccctcctcatcatgggatttcatcacagtaaacaacaatctcacctctgaccaggctgtccaggtatgcaaagataataagtggtgcaaccccttagttattcggtttacagacgccgggagacgggttacttcctggaccacaggacattactggggcttacgtttgtatgtctccggacaagatccagggcttacatttgggatccgactcagataccaaaatctaggaccccgcgtcccaatagggccaaaccccgttctggcagaccaacagccactctccaagcccaaacctgttaagtcgccttcagtcaccaaaccacccagtggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccactaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccaccggtgcgtacgtaccggcgggaacggaaaataggctgctaaacttagtagacggagcctaccaagccctcaacctcaccagtcctgacaaaacccaagagtgctggttgtgtctagtagcgggacccccctactacgaaggggttgccgtcctgggtacctactccaaccatacctctgctccagccaactgctccgtggcctcccaacacaagttgaccctgtccgaagtgaccggacagggactctgcataggagcagttcccaaaacacatcaggccctatgtaataccacccagacaagcagtcgagggtcctattatctagttgcccctacaggtaccatgtgggcttgtagtaccgggcttactccatgcatctccaccaccatactgaaccttaccactgattattgtgttcttgtcgaactctggccaagagtcacctatcattcccccagctatgtttacggcctgtttgagagatccaaccgacacaaaagagaaccggtgtcgttaaccctggccctattattgggtggactaaccatggggggaattgccgctggaataggaacagggactactgctctaatggccactcagcaattccagcagctccaagccgcagtacaggatgatctcagggaggttgaaaaatcaatctctaacctagaaaagtctctcacttccctgtctgaagttgtcctacagaatcgaaggggcctagacttgttatttctaaaagaaggagggctgtgtgctgctctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagccatag

SEQ ID NO: 2 - DNA, 8324 bp, Artificial Sequence
  Retroviral Delivery Vector
cccgaaaagtgccacctgcataatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcacccccctaataggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatctagctagttaattaagccgccaccatggcgcgttcaacgctctcaaaaccccttaaaaataaggttaacccgcgaggccccctaatccccttaattcttctgatgctcagaggggtcagtactgcttcgcccggctccagtcctcatcaagtctataatatcacctgggaggtaaccaatggagatcgggagacggtatgggcaacttctggcaaccaccctctgtggacctggtggcctgaccttaccccagatttatgtatgttagcccaccatggaccatcttattgggggctagaatatcaatcccctttttcttctcccccggggcccccttgttgctcagggggcagcagcccaggctgttccagagactgcgaagaacctttaacctccctcacccctcggtgcaacactgcctggaacagactcaagctagaccagacaactcataaatcaaatgagggattttatgtttgccccgggccccaccgcccccgagaatccaagtcatgtgggggtccagactccttctactgtgcctattggggctgtgagacaaccggtagagcttactggaagccctcctcatcatgggatttcatcacagtaaacaacaatctcacctctgaccaggctgtccaggtatgcaaagataataagtggtgcaaccccttagttattcggtttacagacgccgggagacgggttacttcctggaccacaggacattactggggcttacgtttgtatgtctccggacaagatccagggcttacatttgggatccgactcagataccaaaatctaggaccccgcgtcccaatagggccaaaccccgttctggcagaccaacagccactctccaagcccaaacctgttaagtcgccttcagtcaccaaaccacccagtggggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccactaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccaccggtgcgtacgtaccggcgggaacggaaaataggctgctaaacttagtagacggagcctaccaagccctcaacctcaccagtcctgacaaaacccaagagtgctggttgtgtctagtagcgggacccccctactacgaaggggttgccgtcctgggtacctactccaaccatacctctgctccagccaactgctccgtggcctcccaacacaagttgaccctgtccgaagtgaccggacagggactctgcataggagcagttcccaaaacacatcaggccctatgtaataccacccagacaagcagtcgagggtcctattatctagttgcccctacaggtaccatgtgggcttgtagtaccgggcttactccatgcatctccaccaccatactgaaccttaccactgattattgtgttcttgtcgaactctggccaagagtcacctatcattcccccagctatgtttacggcctgtttgagagatccaaccgacacaaaagagaaccggtgtcgttaaccctggccctattattgggtggactaaccatggggggaattgccgctggaataggaacagggactactgctctaatggccactcagcaattccagcagctccaagccgcagtacaggatgatctcagggaggttgaaaaatcaatctctaacctagaaaagtctctcacttccctgtctgaagttgtcctacagaatcgaaggggcctagacttgttatttctaaaagaaggagggctgtgtgctgctctaaaagaagaatgttgcttctatgcggaccacacaggactagtgagagacagcatggccaaattgagagagaggcttaatcagagacagaaactgtttgagtcaactcaaggatggtttgagggactgtttaacagatccccttggtttaccaccttgatatctaccattatgggacccctcattgtactcctaatgattttgctcttcggaccctgcattcttaatcgattagtccaatttgttaaagacaggatatcagtggtccaggctctagttttgactcaacaatatcaccagctgaagcctatagagtacgagccatagctcgagcggccgccagcacagtggtcgacagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccatcgtcgacgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagttgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttc